SEQUENCE LISTING (17 sequences)

SEQ ID NO: 1 - protein, 572 aa, Clostridium thermocellum
MSVKISGKDYAGMYGPTKGDRVRLADTDLIIEIEEDYTVYGDECKFGGGKSIRDGMGQSPSAARDDKVLDLVITNAIIFDTWGIVKGDIGIKDGKIAGIGKAGNPKVMSGVSEDLIIGASTEVITGEGLIVTPGGIDTHIHFICPQQIETALFSGITTMIGGGTGPADGTNATTCTPGAFNIRKMLEAAEDFPVNLGFLGKGNASFETPLIEQIEAGAIGLKLHEDWGTTPKAIDTCLKVADLFDVQVAIHTDTLNEAGFVENTIAAIAGRTIHTYHTEGAGGGHAPDIIKIASRMNVLPSSTNPTMPFTVNTLDEHLDMLMVCHHLDSKVKEDVAFADSRIRPETIAAEDILHDMGVFSMMSSDSQAMGRVGEVIIRTWQTAHKMKLQRGALPGEKSGCDNIRAKRYLAKYTINPAITHGISQYVGSLEKGKIADLVLWKPAMFGVKPEMIIKGGFIIAGRMGDANASIPTPQPVIYKNMFGAFGKAKYGTCVTFVSKASLENGVVEKMGLQRKVLPVQGCRNISKKYMVHNNATPEIEVDPETYEVKVDGEIITCEPLKVLPMAQRYFLF

SEQ ID NO: 2 - protein, 122 aa, Clostridium thermocellum
MIPGEYIIKNEFITLNDGRRTLNIKVSNTGDRPVQVGSHYHFFEVNRYLEFDRKSAFGMRLDIPSGTAVRFEPGEEKTVQLVEIGGSREIYGLNDLTCGPLDREDLSNVFKKAKELGFKGVE

SEQ ID NO: 3 - protein, 100 aa, Clostridium thermocellum
MHLTPRETEKLMLHYAGELARKRKERGLKLNYPEAVALISAELMEAARDGKTVTELMQYGAKILTRDDVMEGVDAMIHEIQIEATFPDGTKLVTVHNPIR

SEQ ID NO: 4 - protein, 262 aa, Clostridium thermocellum
MKNKFGKESRLYIRAKVSDGKTCLQDSYFTAPFKIAKPFYEGHGGFMNLMVMSASAGVMEGDNYRIEVELDKGARVKLEGQSYQKIHRMKNGTAVQYNSFTLADGAFLDYAPNPTIPFADSAFYSNTECRMEEGSAFIYSEILAAGRVKSGEIFRFREYHSGIKIYYGGELIFLENQFLFPKVQNLEGIGFFEGFTHQASMGFFCKQISDELIDKLCVMLTAMEDVQFGLSKTKKYGFVVRILGNSSDRLESILKLIRNILY

SEQ ID NO: 5 - protein, 153 aa, Clostridium thermocellum
MIVERVLYNIKDIDLEKLEVDFVDIEWYEVQKKILRKLSSNGIEVGIRNSNGEALKEGDVLWQEGNKVLVVRIPYCDCIVLKPQNMYEMGKTCYEMGNRHAPLFIDGDELMTPYDEPLMQALIKCGLSPYKKSCKLTTPLGGNLHGYSHSHSH

SEQ ID NO: 6 - protein, 240 aa, Clostridium thermocellum
MDTPILIPTDMNRIPFFYLLQISDPLFPIGGFTQSYGLETYVQKGIVHDAETSKKYLESYLLNSFLYNDLLAVRLSWEYTQKGNLNKVLELSEVFSASKAPRELRAANEKLGRRFIKILEFVLGENEMFCEMYEKVGRGSVEVSYPVMYGFCTNLLNIGKKEALSAVTYSAASSIINNCAKLVPISQNEGQKILFNAHGIFRRLLERVEELDEEYLGSCCFGFDLRAMQHERLYTRLYIS

SEQ ID NO: 7 - protein, 202 aa, Clostridium thermocellum
MNYVKIGVGGPVGSGKTALIEKLTRILADSYSIGVVTNDIYTKEDAEFLIKNSVLPKERIIGVETGGCPHTAIREDASMNLEAVEELVQRFPDIQIVFIESGGDNLSATFSPELADATIYVIDVAEGDKIPRKGGPGITRSDLLVINKIDLAPYVGASLEVMERDSKKMRGEKPFIFTNLNTNEGVDKIIDWIKKSVLLEGV

SEQ ID NO: 8 - DNA, 1719 bp, Clostridium thermocellum
atgagtgtaaaaataagcggcaaagattatgccggtatgtatggcccgacaaaaggcgacagggtgaggctggcagacacggatctcattattgagattgaggaagattacacggtttatggagatgagtgcaaattcggaggaggtaaatccataagggacggaatgggccagtctccttcggctgcaagagatgacaaggttttggatttggtaattaccaatgccataatctttgacacatgggggattgtaaagggagatataggtataaaagacggaaaaatagccggaatcgggaaggcgggaaatccgaaagtaatgagcggcgtgtcggaggatttaataatcggggcctctaccgaagttattaccggagaaggacttattgtgactccgggaggaattgatacacatatacattttatatgcccccagcagattgagaccgcattgttcagcggtatcacaacaatgattggtggcggaacgggaccggcagacggaaccaatgccaccacttgcacaccgggagcctttaacatccggaaaatgttagaggcggcagaggactttccggtaaatttaggttttttggggaaagggaatgcttcttttgagactcctctgatagaacagattgaagcaggggcgattggcttaaagctccatgaggattggggaaccacacccaaggctatagatacatgcctgaaagttgcggatctttttgatgtacaggtggctatacataccgatacactgaacgaggcaggatttgtagagaatactatagcggctatagccggaaggacaattcacacttaccataccgagggagcgggcggcgggcacgcaccggacataattaaaattgcatcacgcatgaatgtactgccctcgtctaccaatcccaccatgccttttaccgtcaatacattggatgaacatctcgatatgcttatggtatgccatcatcttgacagcaaggtaaaagaggacgttgcttttgccgattcgaggatccggcctgagacaatagccgcagaagacatactgcacgatatgggagtattcagcatgatgagttccgattcccaggccatgggacgcgtgggagaggttattataaggacctggcagactgcacataaaatgaagcttcaaagaggtgccctgccgggggaaaagagcggctgtgacaatataagggctaaaagataccttgccaagtataccataaaccctgctataacccatggaatttcacagtatgtgggctccctggagaaagggaaaatagccgacttggtcctctggaagcctgcaatgtttggtgtaaagcctgaaatgattattaagggcggctttataatagccggcaggatgggcgatgcaaatgcgtccatacccacacctcagcctgtaatatataaaaacatgttcggtgccttcggaaaggcaaagtacggaacctgtgtgacttttgtttcaaaggcttcgctggaaaatggcgttgtggaaaagatggggcttcaaagaaaagtgcttccggtccagggatgcaggaatatctcaaaaaaatatatggtacacaacaatgcaacgcctgaaattgaagttgatcctgaaacctatgaggtaaaggtggacggtgagattatcacctgcgaaccattaaaggtcttacccatggcgcagagatatttcttgttttaa

SEQ ID NO: 9 - DNA, 369 bp, Clostridium thermocellum
atgattcctggcgagtacattataaaaaatgagtttatcacattgaatgatggaagaaggactttaaatatcaaggtttcaaatacaggagaccggcccgttcaggtggggtcccactaccatttcttcgaagttaatcggtatcttgagtttgacagaaaaagcgctttcggaatgagactggacattccttcgggtactgcggtaaggtttgagccgggggaggaaaagacagttcaactggttgaaatagggggaagcagagaaatttacggacttaatgatctgacttgcggtccccttgacagagaagatttgtccaatgtgtttaaaaaggcgaaagagctggggttcaagggggtggaataa

SEQ ID NO: 10 - DNA, 303 bp, Clostridium thermocellum
gtgcatttgacgcccagggaaaccgaaaaattgatgcttcattatgccggtgaactggcaagaaaacgaaaagaaagaggtcttaagcttaattatccggaagctgtagcccttataagcgctgaactgatggaggccgcccgggacggaaaaactgtaacggaactgatgcagtatggagcaaagatactgaccagggatgatgtaatggaaggagttgacgccatgatacatgaaattcagatagaggcaactttcccggacggtacaaagcttgttaccgttcacaatcctatacgctag

SEQ ID NO: 11 - DNA, 789 bp, Clostridium thermocellum
atgaagaataaattcggaaaagaaagcaggctgtacataagagcaaaggtttcagacggaaaaacatgccttcaggattcgtatttcacagcaccttttaaaatagccaaacccttttatgaagggcatggcggatttatgaatcttatggttatgtcagcttcagcgggagttatggagggtgacaattacaggattgaagtggaattggacaaaggcgcaagagtgaaactggaaggccagtcctaccagaagattcaccggatgaaaaatggaacggcagtgcagtacaacagttttacccttgcagacggagcgtttttggattatgctcccaaccccaccataccttttgccgactcagcattttattcaaatacagaatgcaggatggaagaaggctcagcctttatctattcggagatactggccgcgggcagggttaagagcggtgaaattttccggttcagggaatatcacagcgggataaagatttattacggcggggaactgatttttcttgaaaatcagttcctttttccaaaagtgcagaatcttgaaggaatcggattttttgaaggttttacacatcaggcgtcaatgggttttttttgtaagcagataagcgatgaacttattgataaactttgtgtaatgcttacggccatggaggatgtccagttcggattgagcaaaacaaagaagtatggctttgttgttcggattctcggaaacagcagtgataggctggaaagtattctaaaactgattagaaatatcctctattag

SEQ ID NO: 12 - DNA, 462 bp, Clostridium thermocellum
atgattgttgaaagagttttgtataatatcaaagatatcgacttggaaaaattggaagttgatttcgtggatattgaatggtatgaagttcaaaaaaaaatactacgcaaattaagttccaacggaattgaagttggaataagaaacagcaacggtgaggctttaaaagaaggagacgtattgtggcaggagggaaataaagttttggttgtaaggattccctattgcgactgtatcgtgctgaagcctcaaaatatgtatgagatgggcaagacttgctatgagatgggaaacagacatgcacctctttttattgatggagatgagctgatgactccctatgatgagccgttgatgcaggcattgataaaatgcgggctttcaccttacaaaaagagctgtaaacttacaacgcccttaggaggtaatcttcatggatactcccattctcattcccactga

SEQ ID NO: 13 - DNA, 723 bp, Clostridium thermocellum
atggatactcccattctcattcccactgatatgaatagaataccctttttttaccttttacagattagcgatccgctgtttccgataggaggttttacccaatcctatgggcttgaaacctatgtgcaaaaagggattgtccatgatgctgaaacttcgaaaaaataccttgaaagctatcttttaaacagctttttgtacaatgatttattggccgtcaggctttcctgggaatatacccaaaaaggaaatttgaataaggtattggaactttcggaagttttttcggcctcaaaggcgccgagggagcttagagcggcaaatgaaaagctcggcaggaggtttataaagatactggaatttgttttgggcgaaaacgaaatgttttgcgaaatgtatgaaaaagtggggagaggaagtgtggaagtttcgtatcctgtaatgtacggtttttgtacaaatcttctcaatatcggaaaaaaggaagcgttgtcggcggttacttatagcgcggcatcttccataataaataactgtgcaaaattggtacctatcagccagaacgaagggcagaagattttattcaatgcccatggcattttccgaaggcttttggaaagagtggaggaactggacgaggaatatctgggaagctgctgctttggatttgacttaagagccatgcagcatgaaaggctctatacaaggctttatatatcctag

SEQ ID NO: 14 - DNA, 609 bp, Clostridium thermocellum
atgaattatgtgaaaatcggcgtgggaggtccggtaggatcgggcaagaccgcccttatagaaaaattgacaagaatattggctgattcttacagcatcggggtggttaccaacgatatatacacaaaagaggacgcggaatttttaataaagaacagtgtacttcccaaagagaggataattggagtggaaaccggcggctgccctcatacggctattcgcgaggatgcttccatgaaccttgaagctgtggaggaactggtacagcggttccctgatattcaaattgtgtttattgaaagcgggggagacaatctttccgcaactttcagtccggaactggccgatgccaccatatatgtcatcgatgtggccgaaggtgacaaaattccccgaaaaggcggcccgggaataacccggtcggatttactggtcataaataaaattgatctggctccatacgtgggagcaagccttgaggtaatggaaagggattcaaagaagatgaggggtgagaaaccttttatattcaccaatttgaatacaaatgaaggtgtggataagattatcgattggattaagaaaagcgtccttttggaaggtgtgtaa

SEQ ID NO: 15 - DNA, 24326 bp, Artificial
  pDest-Ct-urease (pMU1336)
tggagtttgtaatggatgtggccgactatttttacgttatggataaaggccgcatagtaatggagggaaaaacggagggaatcgatcctcatgaaatacaggaaaagattgctatttgataagtatgtcattgataaatatgccataaaattttgcgcctgtaaatttcgttgttaaaaatattacaaaaaaccaaaagcaatgaataagtatttttagacagggaaaataaattttcctttggttatgccaatttatggattaatcaatttaaaagaaggtggtaagagtgcatttgacgcccagggaaaccgaaaaattgatgcttcattatgccggtgaactggcaagaaaacgaaaagaaagaggtcttaagcttaattatccggaagctgtagcccttataagcgctgaactgatggaggccgcccgggacggaaaaactgtaacggaactgatgcagtatggagcaaagatactgaccagggatgatgtaatggaaggagttgacgccatgatacatgaaattcagatagaggcaactttcccggacggtacaaagcttgttaccgttcacaatcctatacgctagagggaggaaggatgtatgattcctggcgagtacattataaaaaatgagtttatcacattgaatgatggaagaaggactttaaatatcaaggtttcaaatacaggagaccggcccgttcaggtggggtcccactaccatttcttcgaagttaatcggtatcttgagtttgacagaaaaagcgctttcggaatgagactggacattccttcgggtactgcggtaaggtttgagccgggggaggaaaagacagttcaactggttgaaatagggggaagcagagaaatttacggacttaatgatctgacttgcggtccccttgacagagaagatttgtccaatgtgtttaaaaaggcgaaagagctggggttcaagggggtggaataacatgagtgtaaaaataagcggcaaagattatgccggtatgtatggcccgacaaaaggcgacagggtgaggctggcagacacggatctcattattgagattgaggaagattacacggtttatggagatgagtgcaaattcggaggaggtaaatccataagggacggaatgggccagtctccttcggctgcaagagatgacaaggttttggatttggtaattaccaatgccataatctttgacacatgggggattgtaaagggagatataggtataaaagacggaaaaatagccggaatcgggaaggcgggaaatccgaaagtaatgagcggcgtgtcggaggatttaataatcggggcctctaccgaagttattaccggagaaggacttattgtgactccgggaggaattgatacacatatacattttatatgcccccagcagattgagaccgcattgttcagcggtatcacaacaatgattggtggcggaacgggaccggcagacggaaccaatgccaccacttgcacaccgggagcctttaacatccggaaaatgttagaggcggcagaggactttccggtaaatttaggttttttggggaaagggaatgcttcttttgagactcctctgatagaacagattgaagcaggggcgattggcttaaagctccatgaggattggggaaccacacccaaggctatagatacatgcctgaaagttgcggatctttttgatgtacaggtggctatacataccgatacactgaacgaggcaggatttgtagagaatactatagcggctatagccggaaggacaattcacacttaccataccgagggagcgggcggcgggcacgcaccggacataattaaaattgcatcacgcatgaatgtactgccctcgtctaccaatcccaccatgccttttaccgtcaatacattggatgaacatctcgatatgcttatggtatgccatcatcttgacagcaaggtaaaagaggacgttgcttttgccgattcgaggatccggcctgagacaatagccgcagaagacatactgcacgatatgggagtattcagcatgatgagttccgattcccaggccatgggacgcgtgggagaggttattataaggacctggcagactgcacataaaatgaagcttcaaagaggtgccctgccgggggaaaagagcggctgtgacaatataagggctaaaagataccttgccaagtataccataaaccctgctataacccatggaatttcacagtatgtgggctccctggagaaagggaaaatagccgacttggtcctctggaagcctgcaatgtttggtgtaaagcctgaaatgattattaagggcggctttataatagccggcaggatgggcgatgcaaatgcgtccatacccacacctcagcctgtaatatataaaaacatgttcggtgccttcggaaaggcaaagtacggaacctgtgtgacttttgtttcaaaggcttcgctggaaaatggcgttgtggaaaagatggggcttcaaagaaaagtgcttccggtccagggatgcaggaatatctcaaaaaaatatatggtacacaacaatgcaacgcctgaaattgaagttgatcctgaaacctatgaggtaaaggtggacggtgagattatcacctgcgaaccattaaaggtcttacccatggcgcagagatatttcttgttttaaactgccggaaggttagtttctctgtaaaaaatttatggtaattgacatttcaaaaaacaattttaaactaaagaaatttttaaataaagaataattttgggaggacttaaaaaaaactcaaaaacataagttgggtgagatgaaatgattgttgaaagagttttgtataatatcaaagatatcgacttggaaaaattggaagttgatttcgtggatattgaatggtatgaagttcaaaaaaaaatactacgcaaattaagttccaacggaattgaagttggaataagaaacagcaacggtgaggctttaaaagaaggagacgtattgtggcaggagggaaataaagttttggttgtaaggattccctattgcgactgtatcgtgctgaagcctcaaaatatgtatgagatgggcaagacttgctatgagatgggaaacagacatgcacctctttttattgatggagatgagctgatgactccctatgatgagccgttgatgcaggcattgataaaatgcgggctttcaccttacaaaaagagctgtaaacttacaacgcccttaggaggtaatcttcatggatactcccattctcattcccactgatatgaatagaataccctttttttaccttttacagattagcgatccgctgtttccgataggaggttttacccaatcctatgggcttgaaacctatgtgcaaaaagggattgtccatgatgctgaaacttcgaaaaaataccttgaaagctatcttttaaacagctttttgtacaatgatttattggccgtcaggctttcctgggaatatacccaaaaaggaaatttgaataaggtattggaactttcggaagttttttcggcctcaaaggcgccgagggagcttagagcggcaaatgaaaagctcggcaggaggtttataaagatactggaatttgttttgggcgaaaacgaaatgttttgcgaaatgtatgaaaaagtggggagaggaagtgtggaagtttcgtatcctgtaatgtacggtttttgtacaaatcttctcaatatcggaaaaaaggaagcgttgtcggcggttacttatagcgcggcatcttccataataaataactgtgcaaaattggtacctatcagccagaacgaagggcagaagattttattcaatgcccatggcattttccgaaggcttttggaaagagtggaggaactggacgaggaatatctgggaagctgctgctttggatttgacttaagagccatgcagcatgaaaggctctatacaaggctttatatatcctagtgttaataatcctgtactacattgttatttatcttcttaaggaaggtggagcttatgaattatgtgaaaatcggcgtgggaggtccggtaggatcgggcaagaccgcccttatagaaaaattgacaagaatattggctgattcttacagcatcggggtggttaccaacgatatatacacaaaagaggacgcggaatttttaataaagaacagtgtacttcccaaagagaggataattggagtggaaaccggcggctgccctcatacggctattcgcgaggatgcttccatgaaccttgaagctgtggaggaactggtacagcggttccctgatattcaaattgtgtttattgaaagcgggggagacaatctttccgcaactttcagtccggaactggccgatgccaccatatatgtcatcgatgtggccgaaggtgacaaaattccccgaaaaggcggcccgggaataacccggtcggatttactggtcataaataaaattgatctggctccatacgtgggagcaagccttgaggtaatggaaagggattcaaagaagatgaggggtgagaaaccttttatattcaccaatttgaatacaaatgaaggtgtggataagattatcgattggattaagaaaagcgtccttttggaaggtgtgtaaattatgaagaataaattcggaaaagaaagcaggctgtacataagagcaaaggtttcagacggaaaaacatgccttcaggattcgtatttcacagcaccttttaaaatagccaaacccttttatgaagggcatggcggatttatgaatcttatggttatgtcagcttcagcgggagttatggagggtgacaattacaggattgaagtggaattggacaaaggcgcaagagtgaaactggaaggccagtcctaccagaagattcaccggatgaaaaatggaacggcagtgcagtacaacagttttacccttgcagacggagcgtttttggattatgctcccaaccccaccataccttttgccgactcagcattttattcaaatacagaatgcaggatggaagaaggctcagcctttatctattcggagatactggccgcgggcagggttaagagcggtgaaattttccggttcagggaatatcacagcgggataaagatttattacggcggggaactgatttttcttgaaaatcagttcctttttccaaaagtgcagaatcttgaaggaatcggattttttgaaggttttacacatcaggcgtcaatgggttttttttgtaagcagataagcgatgaacttattgataaactttgtgtaatgcttacggccatggaggatgtccagttcggattgagcaaaacaaagaagtatggctttgttgttcggattctcggaaacagcagtgataggctggaaagtattctaaaactgattagaaatatcctctattagtaaaaataaacactatttttggttatgaaaatcagaactaaatgtttttggcagtataaaactgtaaaaacggtttaaaaaaagaaagtgtacaagcattgaaaaatatcaacgttaaaaaagttgtaatttagagatgagccggttgttgaaaagttgaatgcccaaatcccgttaagttatatcttaatcggaaaaaagaataaaagaaattcgatttatgataaaataccttgacaattttggattacagctgtaagatataattagacttacaattgtaatctaaaatggaggggcaattatgaaagcagagtctcaaatcacagaagcggaactggaagttatgaaaattctttgggagtatggaaaggccaccagttctcagatcatagtgactggatatgttgtgttttacagtattatgtagtctgttttttatgcaaaatctaatttaatatattgatatttatatcattttacgtttctcgttcagctttcttgtacaaagtggtaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcggatccgcaagagattatatcgagtgcctttaagaaggctaaaaattacgaagatgtgatacacaaaaaggcaaaagattacggcaaaaacataccggatagtcaagttaaaggagtattgaaacagatagagattactgccttaaaccatgtagacaagattgtcgctgctgaaaagacgatgcagatagattccctcgtgaagaaaaatatgtcttatgatatgatggatgcattgcaggatatagagaaggatttgataaatcagcagatgttctacaacgaaaatctaataaacataaccaatccgtatgtgaggcagatattcactcagatgagggatgatgagatgcgatttatcactatcatacagcagaacatagaatcgttaaagtcaaagccgactgagcccaacagcatagtatatacgacgccgagggaaaataaatgaaagtagctattataggagcaggctcggcaggcttaactgcagctataaggcttgaatcttatgggataaagcctgatatatttgagagaaaatcgaaagtcggcgatgcttttaaccatgtaggaggacttttaaatgtcataaataggccaataaatgatcctttagagtatctaaaaaataactttgatgtagctattgcaccgcttaacaacatagacaagattgtgatgcatgggccaacagtcactcgcacaattaaaggcagaaggcttggatactttatgctgaaagggcaaggagaattgtcagtagaaagccaactatacaagaaattaaagacaaatgtcaattttgatgtccacgcagactacaagaacctaaaggaaatttatgattatgtcattgtagcaactggaaatcatcagataccaaatgagttaggatgttggcagacgcttgttgatacgaggcttaaaattgctgaggtaatcggtaaattcgacccgtctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaattggagtttgtaatggatgtggccgactatttttacgttatggataaaggccgcatagtaatggagggaaaaacggagggaatcgatcctcatgaaatacaggaaaagattgctatttgataagtatgtcattgataaatatgccataaaattttgcgcctgtaaatttcgttgttaaaaatattacaaaaaaccaaaagcaatgaataagtatttttagacagggaaaataaattttcctttggttatgccaatttatggattaatcaatttaaaagaaggtggtaagagtgcatttgacgcccagggaaaccgaaaaattgatgcttcattatgccggtgaactggcaagaaaacgaaaagaaagaggtcttaagcttaattatccggaagctgtagcccttataagcgctgaactgatggaggccgcccgggacggaaaaactgtaacggaactgatgcagtatggagcaaagatactgaccagggatgatgtaatggaaggagttgacgccatgatacatgaaattcagatagaggcaactttcccggacggtacaaagcttgttaccgttcacaatcctatacgctagagggaggaaggatgtatgattcctggcgagtacattataaaaaatgagtttatcacattgaatgatggaagaaggactttaaatatcaaggtttcaaatacaggagaccggcccgttcaggtggggtcccactaccatttcttcgaagttaatcggtatcttgagtttgacagaaaaagcgctttcggaatgagactggacattccttcgggtactgcggtaaggtttgagccgggggaggaaaagacagttcaactggttgaaatagggggaagcagagaaatttacggacttaatgatctgacttgcggtccccttgacagagaagatttgtccaatgtgtttaaaaaggcgaaagagctggggttcaagggggtggaataacatgagtgtaaaaataagcggcaaagattatgccggtatgtatggcccgacaaaaggcgacagggtgaggctggcagacacggatctcattattgagattgaggaagattacacggtttatggagatgagtgcaaattcggaggaggtaaatccataagggacggaatgggccagtctccttcggctgcaagagatgacaaggttttggatttggtaattaccaatgccataatctttgacacatgggggattgtaaagggagatataggtataaaagacggaaaaatagccggaatcgggaaggcgggaaatccgaaagtaatgagcggcgtgtcggaggatttaataatcggggcctctaccgaagttattaccggagaaggacttattgtgactccgggaggaattgatacacatatacattttatatgcccccagcagattgagaccgcattgttcagcggtatcacaacaatgattggtggcggaacgggaccggcagacggaaccaatgccaccacttgcacaccgggagcctttaacatccggaaaatgttagaggcggcagaggactttccggtaaatttaggttttttggggaaagggaatgcttcttttgagactcctctgatagaacagattgaagcaggggcgattggcttaaagctccatgaggattggggaaccacacccaaggctatagatacatgcctgaaagttgcggatctttttgatgtacaggtggctatacataccgatacactgaacgaggcaggatttgtagagaatactatagcggctatagccggaaggacaattcacacttaccataccgagggagcgggcggcgggcacgcaccggacataattaaaattgcatcacgcatgaatgtactgccctcgtctaccaatcccaccatgccttttaccgtcaatacattggatgaacatctcgatatgcttatggtatgccatcatcttgacagcaaggtaaaagaggacgttgcttttgccgattcgaggatccggcctgagacaatagccgcagaagacatactgcacgatatgggagtattcagcatgatgagttccgattcccaggccatgggacgcgtgggagaggttattataaggacctggcagactgcacataaaatgaagcttcaaagaggtgccctgccgggggaaaagagcggctgtgacaatataagggctaaaagataccttgccaagtataccataaaccctgctataacccatggaatttcacagtatgtgggctccctggagaaagggaaaatagccgacttggtcctctggaagcctgcaatgtttggtgtaaagcctgaaatgattattaagggcggctttataatagccggcaggatgggcgatgcaaatgcgtccatacccacacctcagcctgtaatatataaaaacatgttcggtgccttcggaaaggcaaagtacggaacctgtgtgacttttgtttcaaaggcttcgctggaaaatggcgttgtggaaaagatggggcttcaaagaaaagtgcttccggtccagggatgcaggaatatctcaaaaaaatatatggtacacaacaatgcaacgcctgaaattgaagttgatcctgaaacctatgaggtaaaggtggacggtgagattatcacctgcgaaccattaaaggtcttacccatggcgcagagatatttcttgttttaaactgccggaaggttagtttctctgtaaaaaatttatggtaattgacatttcaaaaaacaattttaaactaaagaaatttttaaataaagaataattttgggaggacttaaaaaaaactcaaaaacataagttgggtgagatgaaatgattgttgaaagagttttgtataatatcaaagatatcgacttggaaaaattggaagttgatttcgtggatattgaatggtatgaagttcaaaaaaaaatactacgcaaattaagttccaacggaattgaagttggaataagaaacagcaacggtgaggctttaaaagaaggagacgtattgtggcaggagggaaataaagttttggttgtaaggattccctattgcgactgtatcgtgctgaagcctcaaaatatgtatgagatgggcaagacttgctatgagatgggaaacagacatgcacctctttttattgatggagatgagctgatgactccctatgatgagccgttgatgcaggcattgataaaatgcgggctttcaccttacaaaaagagctgtaaacttacaacgcccttaggaggtaatcttcatggatactcccattctcattcccactgatatgaatagaataccctttttttaccttttacagattagcgatccgctgtttccgataggaggttttacccaatcctatgggcttgaaacctatgtgcaaaaagggattgtccatgatgctgaaacttcgaaaaaataccttgaaagctatcttttaaacagctttttgtacaatgatttattggccgtcaggctttcctgggaatatacccaaaaaggaaatttgaataaggtattggaactttcggaagttttttcggcctcaaaggcgccgagggagcttagagcggcaaatgaaaagctcggcaggaggtttataaagatactggaatttgttttgggcgaaaacgaaatgttttgcgaaatgtatgaaaaagtggggagaggaagtgtggaagtttcgtatcctgtaatgtacggtttttgtacaaatcttctcaatatcggaaaaaaggaagcgttgtcggcggttacttatagcgcggcatcttccataataaataactgtgcaaaattggtacctatcagccagaacgaagggcagaagattttattcaatgcccatggcattttccgaaggcttttggaaagagtggaggaactggacgaggaatatctgggaagctgctgctttggatttgacttaagagccatgcagcatgaaaggctctatacaaggctttatatatcctagtgttaataatcctgtactacattgttatttatcttcttaaggaaggtggagcttatgaattatgtgaaaatcggcgtgggaggtccggtaggatcgggcaagaccgcccttatagaaaaattgacaagaatattggctgattcttacagcatcggggtggttaccaacgatatatacacaaaagaggacgcggaatttttaataaagaacagtgtacttcccaaagagaggataattggagtggaaaccggcggctgccctcatacggctattcgcgaggatgcttccatgaaccttgaagctgtggaggaactggtacagcggttccctgatattcaaattgtgtttattgaaagcgggggagacaatctttccgcaactttcagtccggaactggccgatgccaccatatatgtcatcgatgtggccgaaggtgacaaaattccccgaaaaggcggcccgggaataacccggtcggatttactggtcataaataaaattgatctggctccatacgtgggagcaagccttgaggtaatggaaagggattcaaagaagatgaggggtgagaaaccttttatattcaccaatttgaatacaaatgaaggtgtggataagattatcgattggattaagaaaagcgtccttttggaaggtgtgtaaattatgaagaataaattcggaaaagaaagcaggctgtacataagagcaaaggtttcagacggaaaaacatgccttcaggattcgtatttcacagcaccttttaaaatagccaaacccttttatgaagggcatggcggatttatgaatcttatggttatgtcagcttcagcgggagttatggagggtgacaattacaggattgaagtggaattggacaaaggcgcaagagtgaaactggaaggccagtcctaccagaagattcaccggatgaaaaatggaacggcagtgcagtacaacagttttacccttgcagacggagcgtttttggattatgctcccaaccccaccataccttttgccgactcagcattttattcaaatacagaatgcaggatggaagaaggctcagcctttatctattcggagatactggccgcgggcagggttaagagcggtgaaattttccggttcagggaatatcacagcgggataaagatttattacggcggggaactgatttttcttgaaaatcagttcctttttccaaaagtgcagaatcttgaaggaatcggattttttgaaggttttacacatcaggcgtcaatgggttttttttgtaagcagataagcgatgaacttattgataaactttgtgtaatgcttacggccatggaggatgtccagttcggattgagcaaaacaaagaagtatggctttgttgttcggattctcggaaacagcagtgataggctggaaagtattctaaaactgattagaaatatcctctattagtaaaaataaacactatttttggttatgaaaatcagaactaaatgtttttggcagtataaaactgtaaaaacggtttaaaaaaagaaagtgtacaagcattgaaaaatatcaacgttaaaaaagttgtaatttagagatgagccggttgttgaaaagttgaatgcccaaatcccgttaagttatatcttaatcggaaaaaagaataaaagaaattcgatttatgataaaataccttgacaattttggattacagctgtaagatataattagacttacaattgtaatctaaaatggaggggcaattatgaaagcagagtctcaaatcacagaagcggaactggaagttatgaaaattctttgggagtatggaaaggccaccagttctcagatcatagtgactggatatgttgtgttttacagtattatgtagtctgttttttatgcaaaatctaatttaatatattgatatttatatcattttacgtttctcgttcagctttcttgtacaaagtggtaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcggatccgcaagagattatatcgagtgcctttaagaaggctaaaaattacgaagatgtgatacacaaaaaggcaaaagattacggcaaaaacataccggatagtcaagttaaaggagtattgaaacagatagagattactgccttaaaccatgtagacaagattgtcgctgctgaaaagacgatgcagatagattccctcgtgaagaaaaatatgtcttatgatatgatggatgcattgcaggatatagagaaggatttgataaatcagcagatgttctacaacgaaaatctaataaacataaccaatccgtatgtgaggcagatattcactcagatgagggatgatgagatgcgatttatcactatcatacagcagaacatagaatcgttaaagtcaaagccgactgagcccaacagcatagtatatacgacgccgagggaaaataaatgaaagtagctattataggagcaggctcggcaggcttaactgcagctataaggcttgaatcttatgggataaagcctgatatatttgagagaaaatcgaaagtcggcgatgcttttaaccatgtaggaggacttttaaatgtcataaataggccaataaatgatcctttagagtatctaaaaaataactttgatgtagctattgcaccgcttaacaacatagacaagattgtgatgcatgggccaacagtcactcgcacaattaaaggcagaaggcttggatactttatgctgaaagggcaaggagaattgtcagtagaaagccaactatacaagaaattaaagacaaatgtcaattttgatgtccacgcagactacaagaacctaaaggaaatttatgattatgtcattgtagcaactggaaatcatcagataccaaatgagttaggatgttggcagacgcttgttgatacgaggcttaaaattgctgaggtaatcggtaaattcgacccgtctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggctcatcgaggtatccaagcgattcaatagtaacagtccttgtatgccctctttctttatcacgatatccatctgcaatagataggtatattcttccggaactgcgtctacttttctttaaatacacattaaactcccccaataaaattcaatataactatattataccacaatccataataatccgcaaccaaaatatgacaaaaatttaaaaaaattttacccaaaatcgttagtaaaattgctggttccgggttacgctacataaaattttgctgcaaaactagggtaaaaaaaatacaaaccatgcgtcaatagaaattgacggcagtatattaaagcagtataatgaatatatggaaaaacaaaagggcaatataatattaaaagggaaatataaacctgaatataaggaaaagttgcttaatttagccaaattttttactgataatggctttgttcctactgaacatgcattgaatgaaatacttgggaaaacagcttctggaagattgccagatgacaaacagatgttattggatgtattacaaaatggtgaaaattatattgaacctaatggcaatatagtcaggtataaaaatggcatatcaatacatatcgataaagaacatggctggataattactataactccaaggaaacgaatagtaaaggaatggaggcgaattaatgagtaatgtcgcaatgcaattaatagaaatttgtcggaaatatgtaaataataatttaaacataaatgaatttatcgaagactttcaagtgctttatgaacaaaagcaagatttattgacagatgaagaaatgagcttgtttgatgatatttatatggcttgtgaatactatgaacaggatgaaaatataagaaatgaatatcacttgtatattggagaaaatgaattaagacaaaaagtgcaaaaacttgtaaaaaagttagcagcataataaaccgctaaggcatgatagctaaaggagtcgtgactaagaacgtcaaagtaattaacaatacagctatttttctcatgcttttacccctttcataaaatttaattttatcgttatcataaaaaattatagacgttatattgcttgccgggatatagtgctgggcattcgttggtgcaaaatgttcggagtaaggtggatattgatttgcatgttgatctattgcattgaaatgattagttatccgtaaatattaattaatcatatcataaattaattatatcataattgttttgacgaatgaaggtttttggataaattatcaagtaaaggaacgctaaaaattttggcgtaaaatatcaaaatgaccacttgaattaatatggtaaagtagatataatattttggtaaacatgccttcagcaaggttagattagctgtttccgtataaattaaccgtatggtaaaacggcagtcagaaaaataagtcataagattccgttatgaaaatatacttcggtagttaataataagagatatgaggtaagagatacaagataagagatataaggtacgaatgtataagatggtgcttttaggcacactaaataaaaaacaaataaacgaaaattttaaggaggacgaaagacaagtttgtacaaaaaagctgaacgagaaacgtaaaatgatataaatatcaatatattaaattagattttgcataaaaaacagactacataatactgtaaaacacaacatatccagtcactatg

SEQ ID NO: 16 - DNA, 18382 bp, Artificial
  pMetE_fix_A (pMU1728)
ccgctcccggcggatttgtcctactcaggagagcgttcaccgacaaacaacagataaaacgaaaggcccagtctttcgactgagcctttcgttttatttgatgcctgggcgatcgtacttactgtttccccttctttaggcaatttgcttgatacaccaacttgtattcttgttggatcatgtattaatattactttgcctttaaatctattacttgatatgtcgtatacttcaattgtgttatcatgagaatttgtaaaatttaatatatttttattgctactgcctgtagcgatattattagaatttttcatgatttcatctattttactctgaggcaagaataatgtaactatatatttatgactaaaagttgtcattgcagatgtaactaatgtatttcttatatttgcgaatggcccataaaatatcaatacaggaattacaataattgataatatgaattcaaaaactaaatatacaataattcttttcgtcaaaatcatatttctcatagataactttcattcctttcatttataaacggcatttatttttagtttaagttttttgggtgtcccatgttgtacatggtagttattcatagtatcctctgtaatatattagcataaaaaatattcaggtatcaacaggaatttaaaaaattttcaaaaaatatattgactttataggtaaaccgcattatattaaataacatagtgttgcctattatttgctaaaagtattgtcatgtattgtaaaaaatctcattttagcttaatatatatttgtaattatatagtgtcggcttaaacatttgtttgatataattattaataacaaaagttatattgattgggatggtagttatgattcagttaactgatacggaaattaaaaaaaggtgtgaaaatgatagtgtctataaaagaggcattgaatattatttggcaggtaggatacacaattttacatacaacaaagctggcactgtatttcaagcttttgtgatgggcacatctttgtacagggtgatgatacaaaagtatcacggtgagttgtacacaagctgtacgagtcgtgactaagaacgtcaaagtaattaacaatacagctatttttctcatgcttttacccctttcataaaatttaattttatcgttatcataaaaaattatagacgttatattgcttgccgggatatagtgctgggcattcgttggtgcaaaatgttcggagtaaggtggatattgatttgcatgttgatctattgcattgaaatgattagttatccgtaaatattaattaatcatatcataaattaattatatcataattgttttgacgaatgaaggtttttggataaattatcaagtaaaggaacgctaaaaattttggcgtaaaatatcaaaatgaccacttgaattaatatggtaaagtagatataatattttggtaaacatgccttcagcaaggttagattagctgtttccgtataaattaaccgtatggtaaaacggcagtcagaaaaataagtcataagattccgttatgaaaatatacttcggtagttaataataagagatatgaggtaagagatacaagataagagatataaggtacgaatgtataagatggtgcttttaggcacactaaataaaaaacaaataaacgaaaattttaaggaggacgaaagatgatttcagttgtcggttttccaagaataggacaaaatagagagcttaaaaaatgggttgagagctatctggacaaaaatctttcaaaagaagagctcattcaaaactcaaaaaacttaaaaaagactcactggcaacttcaaaaagagtatggtgttgacctgatatcatcaaatgacttttcgctttacgacacttttttagaccatgcaatgcttgttggcgcaatacccgaggaatacaaggcggttttctcagatgatctcgagctctactttgcgcttgcaaagggatatcaagaccaaaacattgatcttaaagctttgcctatgaaaaagtggttctttacaaactaccactatcttgtgcctgaaatcactgaaaacaccaaatttgagctttcatcaacaaaaccttttgatgaatttgtcgaagcactttcaataggagttaagacaaaaccggcaataatcggtgctctgacatttttaaagctttccaaaaaatcaaatgtggatatgtacgacaaatctttctgggaaaagctgcttgatgtatatattcaaatactaaaaaggtttgaagagttaggtagcgagtttgttcagatagatgaaccgatacttgtcacagacttaagtacaaaagacatagaattttttgaagatttttatcgcagtcttcttcttcataaaggaaagctgaaggtacttcttcagacctattttggagatgtcagagactgcttcgaaaagataatctcccttgactttgacgcaattggccttgactttgttgatggaaagttcaatttagagctcattaaaaaatttggttttccacaggataagctcctggttgctggagttgtaaatggcagaaatgtgtttaaaaacaactacaaaaatacgcttgagcttttaaatatgctctcctcatttgttgacaagaaaaatattgtaatttcaacatcatgttccttactctttgtgccatactctttgaagttcgaaacacagcttgacagcaataaaaagaagtttttagcgtttgctgaggaaaagctaaaagagctgtctgagcttaagcttttgttctctcaagaaagctttaccgcaaacagcatctatgttcaaaatgttcagctttttgaagagctgaataaaaacaaactatcagatgttagcacagctgtaagtggtcttacagacgatgattttgaaagaaaaccctgttttgaagagagaatcaagcttcaaaaagaggttttgaacttgccacagcttccgacaacaacaattgggtcattcccgcaaaccccggacgtgagggctgctcgaagcaagcttaaaaaaggtgaaataacacttgaagaatataaaaactttataaaatctaagattgaaagagtaataaagcttcaagaagaaatcgggcttgatgtccttgtccacggcgaatacgaaagaaatgacatggtagagtttttcggtgaaaacttggaagggtttttaatcactcaaaacggttgggttcagtcatatggtacaagatgtgtaaaacctcctataatattttctgacattaaaagaaaaaaatcactcacagtggaatatataaaatacgcacaaagcttgacttcgaagcctgtaaaagggatcttgacaggaccagtgacaatcctcaactggtcatttgtgcgcgaagatataccattgaaagatgtagcttttcagcttgctcttgcaataaaagaagaggttttggagcttgaaagagaaggtgtaaagattattcagattgacgaggcagcactgattgaaaagcttccgctcaggcgctgccagcacagtagctatttgtcatgggcgataaaagcattcaggctcacatgttcaaaagtaaaaccagaaactcaaattcatactcatatgtgttacagcaactttgatgagcttttagatgaaatagcaaagatggatgtggacgttataacttttgaggcagctaaatctgattttacattgctcgacagcataaacaaaagtagtttaaaagcagaggtaggtcctggcgtgtttgacgtgcattcacctcgaattgtatcaaaggaagagatgaaaaagctcatattaaagatgatagaaaaggttgggaaagacaggctgtgggtaaaccctgactgcggtcttaaaaccagaaaggaagaagaagttttgcctaccttgcaaaacatggtgcttgcagcgtgggaagtcagaaataacttataatggagtttgtaatggatgtggccgactatttttacgttatggataaaggccgcatagtaatggagggaaaaacggagggaatcgatcctcatgaaatacaggaaaagattgctatttgataagtatgtcattgataaatatgccataaaattttgcgcctgtaaatttcgttgttaaaaatattacaaaaaaccaaaagcaatgaataagtatttttagacagggaaaataaattttcctttggttatgccaatttatggattaatcaatttaaaagaaggtggtaagagtgcatttgacgcccagggaaaccgaaaaattgatgcttcattatgccggtgaactggcaagaaaacgaaaagaaagaggtcttaagcttaattatccggaagctgtagcccttataagcgctgaactgatggaggccgcccgggacggaaaaactgtaacggaactgatgcagtatggagcaaagatactgaccagggatgatgtaatggaaggagttgacgccatgatacatgaaattcagatagaggcaactttcccggacggtacaaagcttgttaccgttcacaatcctatacgctagagggaggaaggatgtatgattcctggcgagtacattataaaaaatgagtttatcacattgaatgatggaagaaggactttaaatatcaaggtttcaaatacaggagaccggcccgttcaggtggggtcccactaccatttcttcgaagttaatcggtatcttgagtttgacagaaaaagcgctttcggaatgagactggacattccttcgggtactgcggtaaggtttgagccgggggaggaaaagacagttcaactggttgaaatagggggaagcagagaaatttacggacttaatgatctgacttgcggtccccttgacagagaagatttgtccaatgtgtttaaaaaggcgaaagagctggggttcaagggggtggaataacatgagtgtaaaaataagcggcaaagattatgccggtatgtatggcccgacaaaaggcgacagggtgaggctggcagacacggatctcattattgagattgaggaagattacacggtttatggagatgagtgcaaattcggaggaggtaaatccataagggacggaatgggccagtctccttcggctgcaagagatgacaaggttttggatttggtaattaccaatgccataatctttgacacatgggggattgtaaagggagatataggtataaaagacggaaaaatagccggaatcgggaaggcgggaaatccgaaagtaatgagcggcgtgtcggaggatttaataatcggggcctctaccgaagttattaccggagaaggacttattgtgactccgggaggaattgatacacatatacattttatatgcccccagcagattgagaccgcattgttcagcggtatcacaacaatgattggtggcggaacgggaccggcagacggaaccaatgccaccacttgcacaccgggagcctttaacatccggaaaatgttagaggcggcagaggactttccggtaaatttaggttttttggggaaagggaatgcttcttttgagactcctctgatagaacagattgaagcaggggcgattggcttaaagctccatgaggattggggaaccacacccaaggctatagatacatgcctgaaagttgcggatctttttgatgtacaggtggctatacataccgatacactgaacgaggcaggatttgtagagaatactatagcggctatagccggaaggacaattcacacttaccataccgagggagcgggcggcgggcacgcaccggacataattaaaattgcatcacgcatgaatgtactgccctcgtctaccaatcccaccatgccttttaccgtcaatacattggatgaacatctcgatatgcttatggtatgccatcatcttgacagcaaggtaaaagaggacgttgcttttgccgattcgaggatccggcctgagacaatagccgcagaagacatactgcacgatatgggagtattcagcatgatgagttccgattcccaggccatgggacgcgtgggagaggttattataaggacctggcagactgcacataaaatgaagcttcaaagaggtgccctgccgggggaaaagagcggctgtgacaatataagggctaaaagataccttgccaagtataccataaaccctgctataacccatggaatttcacagtatgtgggctccctggagaaagggaaaatagccgacttggtcctctggaagcctgcaatgtttggtgtaaagcctgaaatgattattaagggcggctttataatagccggcaggatgggcgatgcaaatgcgtccatacccacacctcagcctgtaatatataaaaacatgttcggtgccttcggaaaggcaaagtacggaacctgtgtgacttttgtttcaaaggcttcgctggaaaatggcgttgtggaaaagatggggcttcaaagaaaagtgcttccggtccagggatgcaggaatatctcaaaaaaatatatggtacacaacaatgcaacgcctgaaattgaagttgatcctgaaacctatgaggtaaaggtggacggtgagattatcacctgcgaaccattaaaggtcttacccatggcgcagagatatttcttgttttaaactgccggaaggttagtttctctgtaaaaaatttatggtaattgacatttcaaaaaacaattttaaactaaagaaatttttaaataaagaataattttgggaggacttaaaaaaaactcaaaaacataagttgggtgagatgaaatgattgttgaaagagttttgtataatatcaaagatatcgacttggaaaaattggaagttgatttcgtggatattgaatggtatgaagttcaaaaaaaaatactacgcaaattaagttccaacggaattgaagttggaataagaaacagcaacggtgaggctttaaaagaaggagacgtattgtggcaggagggaaataaagttttggttgtaaggattccctattgcgactgtatcgtgctgaagcctcaaaatatgtatgagatgggcaagacttgctatgagatgggaaacagacatgcacctctttttattgatggagatgagctgatgactccctatgatgagccgttgatgcaggcattgataaaatgcgggctttcaccttacaaaaagagctgtaaacttacaacgcccttaggaggtaatcttcatggatactcccattctcattcccactgatatgaatagaataccctttttttaccttttacagattagcgatccgctgtttccgataggaggttttacccaatcctatgggcttgaaacctatgtgcaaaaagggattgtccatgatgctgaaacttcgaaaaaataccttgaaagctatcttttaaacagctttttgtacaatgatttattggccgtcaggctttcctgggaatatacccaaaaaggaaatttgaataaggtattggaactttcggaagttttttcggcctcaaaggcgccgagggagcttagagcggcaaatgaaaagctcggcaggaggtttataaagatactggaatttgttttgggcgaaaacgaaatgttttgcgaaatgtatgaaaaagtggggagaggaagtgtggaagtttcgtatcctgtaatgtacggtttttgtacaaatcttctcaatatcggaaaaaaggaagcgttgtcggcggttacttatagcgcggcatcttccataataaataactgtgcaaaattggtacctatcagccagaacgaagggcagaagattttattcaatgcccatggcattttccgaaggcttttggaaagagtggaggaactggacgaggaatatctgggaagctgctgctttggatttgacttaagagccatgcagcatgaaaggctctatacaaggctttatatatcctagtgttaataatcctgtactacattgttatttatcttcttaaggaaggtggagcttatgaattatgtgaaaatcggcgtgggaggtccggtaggatcgggcaagaccgcccttatagaaaaattgacaagaatattggctgattcttacagcatcggggtggttaccaacgatatatacacaaaagaggacgcggaatttttaataaagaacagtgtacttcccaaagagaggataattggagtggaaaccggcggctgccctcatacggctattcgcgaggatgcttccatgaaccttgaagctgtggaggaactggtacagcggttccctgatattcaaattgtgtttattgaaagcgggggagacaatctttccgcaactttcagtccggaactggccgatgccaccatatatgtcatcgatgtggccgaaggtgacaaaattccccgaaaaggcggcccgggaataacccggtcggatttactggtcataaataaaattgatctggctccatacgtgggagcaagccttgaggtaatggaaagggattcaaagaagatgaggggtgagaaaccttttatattcaccaatttgaatacaaatgaaggtgtggataagattatcgattggattaagaaaagcgtccttttggaaggtgtgtaaattatgaagaataaattcggaaaagaaagcaggctgtacataagagcaaaggtttcagacggaaaaacatgccttcaggattcgtatttcacagcaccttttaaaatagccaaacccttttatgaagggcatggcggatttatgaatcttatggttatgtcagcttcagcgggagttatggagggtgacaattacaggattgaagtggaattggacaaaggcgcaagagtgaaactggaaggccagtcctaccagaagattcaccggatgaaaaatggaacggcagtgcagtacaacagttttacccttgcagacggagcgtttttggattatgctcccaaccccaccataccttttgccgactcagcattttattcaaatacagaatgcaggatggaagaaggctcagcctttatctattcggagatactggccgcgggcagggttaagagcggtgaaattttccggttcagggaatatcacagcgggataaagatttattacggcggggaactgatttttcttgaaaatcagttcctttttccaaaagtgcagaatcttgaaggaatcggattttttgaaggttttacacatcaggcgtcaatgggttttttttgtaagcagataagcgatgaacttattgataaactttgtgtaatgcttacggccatggaggatgtccagttcggattgagcaaaacaaagaagtatggctttgttgttcggattctcggaaacagcagtgataggctggaaagtattctaaaactgattagaaatatcctctattagtaaaaataaacactatttttggttatgaaaatcagaactaaatgtttttggcagtataaaactgtaaaaacggtttaaaaaaagaaagtgtacaagcattgaaaaatatcaacgttaaaaaagttgtaatttagagatgagccggttgttgaaaagttgaatgcccaaatcccgttaagttatatcttaatcggaaaaaagaataaaagaaattcgatttatgataaaataccttgacaattttggattacagctgtaagatataattagacttacaattgtaatctaaaatggaggggcaattatgaaagcagagtctcaaatcacagaagcggaactggaagttatgaaaattctttgggagtatggaaaggccaccagttctcagatcgtgcccattgtgaagtggattgtattctacaattaaacctaatacgctcataatatgcgcctttctaaaaaattattaattgtacttattattttataaaaaatatgttaaaatgtaaaatgtgtatacaatatatttcttcttagtaagaggaatgtataaaaataaatattttaaaggaagggacgatcttatgagcattattcaaaacatcattgaaaaagctaaaagcgataaaaagaaaattgttctgccagaaggtgcagaacccaggacattaaaagctgctgaaatagttttaaaagaagggattgcagatttagtgcttcttggaaatgaagatgagataagaaatgctgcaaaagacttggacatatccaaagctgaaatcattgaccctgtaaagtctgaaatgtttgataggtatgctaatgatttctatgagttaaggaagaacaaaggaatcacgttggaaaaagccagagaaacaatcaaggataatatctattttggatgtatgatggttaaagaaggttatgctgatggattggtatctggcgctattcatgctactgcagatttattaagacctgcatttcagataattaaaacggctccaggagcaaagatagtatcaagcttttttataatggaagtgcctaattgtgaatatggtgaaaatggtgtattcttgtttgctgattgtgcggtcaacccatcgcctaatgcagaagaacttgcttctattgccgtacaatctgctaatactgcaaagaatttgttgggctttgaaccaaaagttgccatgctatcattttctacaaaaggtagtgcatcacatgaattagtagataaagtaagaaaagcgacagagatagcaaaagaattgatgccagatgttgctatcgacggtgaattgcaattggatgctgctcttgttaaagaagttgcagagctaaaagcgccgggaagcaaagttgcgggatgtgcaaatgtgcttatattccctgatttacaagctggtaatataggatataagcttgtacagaggttagctaaggcaaatgcaattggacctataacacaaggaatgggtgcaccggttaatgatttatcaagaggatgcagctatagagatattgttgacgtaatagcaacaacagctgtgcaggctcaataaaatgtaaagtatggaggatgaaaattatgaaaatactggttattaattgcggaagttcttcgctaaaatatcaactgattgaatcaactgatggaaatgtgttggcaaaaggccttgctgaaagaatcggcataaatgattccatgttgacacataatgctaacggagaaaaaatcaagataaaaaaagacatgaaagatcacaaagacgcaataaaattggttttagatgctttggtaaacagtgactacggcgttataaaagatatgtctgagatagatgctgtaggacatagagttgttcacggaggagaatcttttacatcatcagttctcataaatgatgaagtgttaaaagcgataacagattgcatagaattagctccactgcacaatcctgctaatatagaaggaattaaagcttgccagcaaatcatgccaaacgttccaatggtggcggtatttgatacagcctttcatcagacaatgcctgattatgcatatctttatccaataccttatgaatactacacaaagtacaggattagaagatatggatttcatggcacatcgcataaatatgtttcaaatagggctgcagagattttgaataaacctattgaagatttgaaaatcataacttgtcatcttggaaatggctccagcattgctgctgtcaaatatggtaaatcaattgacacaagcatgggatttacaccattagaaggtttggctatgggtacacgatctggaagcatagacccatccatcatttcgtatcttatggaaaaagaaaatataagcgctgaagaagtagtaaatatattaaataaaaaatctggtgtttacggtatttcaggaataagcagcgattttagagacttagaagatgccgcctttaaaaatggagatgaaagagctcagttggctttaaatgtgtttgcatatcgagtaaagaagacgattggcgcttatgcagcagctatgggaggcgtcgatgtcattgtatttacagcaggtgttggtgaaaatggtcctgagatacgagaatttatacttgatggattagagtttttagggttcagcttggataaagaaaaaaataaagtcagaggaaaagaaactattatatctacgccgaattcaaaagttagcgtgatggttgtgcctactaatgaagaatacatgattgctaaagatactgaaaagattgtaaagagtataaaatagcattcttgacaaatgtttaccccattagtataattaattttggcaattatattggggtgagaaaatgaaaattgatttatcaaaaattaaaggacataggggccgcagcatcgaagtcaactacgtaaaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttcaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttttagacatctaatcttttctgaagtacatccgcaactgtccatactctgatgttttatatcttttctaaaagttcgctagataggggtcccgagcgcctacgaggaatttgtatcggaagatcaagcgacagatagagcccacaggattgggcaggttaatacagtacaagtcataaagcttataacgcaaggtacaattgaagaaaaaattgtaaagctgcaagagaagaaaaaagagatgataaattctgtcataaatccaggtgaaacgtttataactaagttgagtgaagaagaagtaaaagagctttttgcaatgtgatttaatgatttgcaattgccgattaaggcagttgctttttttatgttacaagattgtaatagaaaattaaggaataattaataaaatttataattttaaattttataatagagatgaggcatgggaggttaagagtataatctatattgataaaagtcactttgtctgggaggctattatgaataaagtgaaactatgtttattaattatcgtaatcttaatacttggtggctgtagtattaaaagtacaaatacagacttaagcaatgataatataattattgataaaacaaatggtaatatacttgatgagttagaggataaaaagacctcatcgattgaaaatgcacatccaatagctgtgcttgatgatggcagaaaagtgtttttgcaggtcaatcctgaagttgacaacagcatttttgttacctcaagtgacagctcaataatttttaaaattaatgctggaatttctaaaaatatttatgatgcaaaagtcatggggaattggatcgtgtatgttgaatccagcaacgatatgacaaaaagcgattgggctttgtatgctaaaaatatagatgacaatcgtcgcatagaaattgataaaggaaatgttgtaaatgcaaaagtaaaaacgcctactttgttaggagcgttgatagctgcatctctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcacctatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatctctaagaaaccattattatcatgacattaacctataaaaaaggcctctcgagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaat

SEQ ID NO: 17 - DNA, 621 bp, Clostridium thermocellum
gagtcgtgactaagaacgtcaaagtaattaacaatacagctatttttctcatgcttttacccctttcataaaatttaattttatcgttatcataaaaaattatagacgttatattgcttgccgggatatagtgctgggcattcgttggtgcaaaatgttcggagtaaggtggatattgatttgcatgttgatctattgcattgaaatgattagttatccgtaaatattaattaatcatatcataaattaattatatcataattgttttgacgaatgaaggtttttggataaattatcaagtaaaggaacgctaaaaattttggcgtaaaatatcaaaatgaccacttgaattaatatggtaaagtagatataatattttggtaaacatgccttcagcaaggttagattagctgtttccgtataaattaaccgtatggtaaaacggcagtcagaaaaataagtcataagattccgttatgaaaatatacttcggtagttaataataagagatatgaggtaagagatacaagataagagatataaggtacgaatgtataagatggtgcttttaggcacactaaataaaaaacaaataaacgaaaattttaaggaggacgaaag

(19) United States
(12) Patent Application Publication
(10) Pub. No.: US 2013/0171708 A1
(43) Pub. Date: Jul. 4, 2013

(54) Heterologous Expression of Urease in Anaerobic, Thermophilic Hosts

(71) Applicants: Sean Covalla, Lebanon, NH (US); Arthur J. Shaw, IV, Grantham, NH (US)

(72) Inventors: Sean Covalla, Lebanon, NH (US); Arthur J. Shaw, IV, Grantham, NH (US)

(21) Appl. No.: 13/514,519

(22) PCT Filed: Dec. 6, 2010
(86) PCT No.: PCT/US10/59120

§ 371 Date: Mar. 22, 2013

Related U.S. Application Data
(60) Provisional application No. 61/267,273, filed on Dec. 7, 2009.

(51) Int. Cl. C12N 15/52; C12P 7/10

(52) U.S. Cl. CPC C12N 15/52; C12P 7/10
USPC 435/165; 435/252.3

(57) ABSTRACT

The invention is directed to the heterologous expression of urease in anaerobic thermophilic hosts, such as Thermoanaerobacterium, Thermoanaerobacter, and other related genera. For example, the anaerobic thermophilic host can be T. saccharolyticum. The host cells express the catalytic subunits of the urease enzyme together with the accessory proteins ureDEFG that facilitate protein folding and nickel activation. The invention further relates to the use of urea as a nitrogen source in the growth of microorganisms involved in consolidated bioprocessing systems.

### BACKGROUND OF THE INVENTION

Urease (EC 3.5.1.5) catalyzes the hydrolysis of urea to CO₂ and ammonia. Bacterial ureases are relatively widespread, and have been well studied, particularly for typing bacteria and the role urease plays in pathogenicity. Ureases have been heterologously expressed in E. coli. Maeda et al., J. Bacteriol. 176:432-442 (1994).

The ability to utilize urea as a nitrogen source has several benefits for a consolidated bioprocessing (CBP) or simultaneous saccharification and fermentation (SSF) configuration. Urea is a low cost nitrogen source that has favorable handling and safety qualities compared to ammonia gas or ammonium hydroxide. In addition, the use of urea does not require active base addition to maintain neutral pH, as is true with ammonium salts. This has benefits for both the large (process) and small (laboratory) scale, where pH control can be technically challenging. Finally, the hydrolysis of urea to ammonia in laboratory media tends to keep the pH at or above 6, which is favorable for a co-culture of certain CBP microorganisms, such as Clostridium thermocellum (C. thermocellum) and Thermoanaerobacterium saccharolyticum (T. saccharolyticum). C. thermocellum carries an active urease enzyme. However, urease enzymes appear to be absent from all known Thermoanaerobacter and Thermoananerbacterium strains. Thus, with respect to the development of robust CBP systems, there is a need in the art for a recombinant Thermoanaerobacter or Thermoananerbacterium microorganism capable of heterologously expressing the urease enzyme.

### BRIEF SUMMARY OF THE INVENTION

The present invention is directed to a recombinant anaerobic, thermophilic host cell, where the anaerobic, thermophilic host heterologously expresses two or three catalytic subunits (α, β and/or γ) and four accessory proteins (D, E, F, and G) of a urease enzyme; where the host cell is capable of catalyzing the hydrolysis of urea to carbon dioxide and ammonia. In certain embodiments, the host is of the genus Thermoanaerobacter or Thermoananerbacterium. In particular embodiments, the host is T. saccharolyticum.

In certain aspects of the invention, the urease catalytic subunits and accessory proteins are derived from an anaerobic, thermophilic organism that natively expresses the urease enzyme. In particular embodiments, the urease catalytic subunits and accessory proteins are derived from Clostridium thermocellum (C. thermocellum).

In certain other aspects of the invention, nickel is properly captured by the metallochaperone ureE and/or the urease apo-enzyme is properly activated by ureD, ureF, and ureG.

The invention is further directed to a method of producing ethanol comprising: (a) culturing the recombinant anaerobic, thermophilic host cell of the invention in the presence of urea as the sole nitrogen source; (b) contacting the anaerobic, thermophilic host cell with lignocellulosic biomass; and (c) recovering the ethanol from the host cell culture. In certain embodiments, the host cell is of the genus Thermoanaerobacter or Thermoananerbacterium. In particular embodiments, the host is T. saccharolyticum.

In certain aspects of the invention, the host cell is co-cultured with a second anaerobic, thermophilic host strain. In particular embodiments, the second anaerobic, thermophilic host strain is C. thermocellum.

In certain other aspects of the invention, the host is cultured in a medium having a pH range of 6 to 9, ideally suited for growth of certain anaerobic thermophilic organisms, such as C. thermocellum as well as species of the genera Thermoanaerbacter or Thermanaerobacterium, such as T. saccharolyticum. In further aspects, the host cell produces increased ethanol titers with utilization of urea as a sole nitrogen source as compared to the levels of ethanol produced with utilization of complex additives or ammonium salts as a nitrogen source.

### BRIEF DESCRIPTION OF THE DRAWINGS/FIGURES

FIG. 1 depicts a schematic diagram of the plasmid constructs used to create the urease⁺ T. saccharolyticum strains M1051 (FIG. 1A) and M1151 (FIG. 1B).

FIG. 2 depicts a graph showing pressure measurements over time for urease and urease⁻ strains of T. saccharolyticum using different nitrogen sources.

FIG. 3 depicts two bar graphs showing the fermentation performance of urease and urease⁺ T. saccharolyticum strains in various growth media.

### DETAILED DESCRIPTION OF THE INVENTION

### Definitions

A “vector,” e.g., a “plasmid” or “YAC” (yeast artificial chromosome) refers to an extrachromosomal element often carrying one or more genes that are not part of the central metabolism of the cell, and is usually in the form of a circular double-stranded DNA molecule. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. Preferably, the plasmids or vectors of the present invention are stable and self-replicating.

An “expression vector” is a vector that is capable of directing the expression of genes to which it is operably associated.

The term “heterologous” as used herein refers to an element of a vector, plasmid or host cell that is derived from a source other than the endogenous source. Thus, for example, a heterologous sequence could be a sequence that is derived from a different gene or plasmid from the same host, from a different strain of host cell, or from an organism of a different taxonomic group (e.g., different kingdom, phylum, class, order, family genus, or species, or any subgroup within one of these classifications). The term “heterologous” is also used synonymously herein with the term “exogenous.”

A “nucleic acid,” “polynucleotide,” or “nucleic acid molecule” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

An “isolated nucleic acid molecule” or “isolated nucleic acid fragment” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A “gene” refers to an assembly of nucleotides that encode a polypeptide, and includes cDNA and genomic DNA nucleic acids. “Gene” also refers to a nucleic acid fragment that expresses a specific protein, including intervening sequences (introns) between individual coding segments (exons), as well as regulatory sequences preceding (5′ non-coding sequences) and following (3′ non-coding sequences) the coding sequence. “Native gene” refers to a gene as found in nature with its own regulatory sequences.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences.

As known in the art, “similarity” between two polypeptides is determined by comparing the amino acid sequence and conserved amino acid substitutes thereto of the polypeptide to the sequence of a second polypeptide.

A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region.

“Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

“Promoter” refers to a DNA fragment capable of controlling the expression of a coding sequence or functional RNA. In general, a coding region is located 3′ to a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of RNA polymerase.

A coding region is “under the control” of transcriptional and translational control elements in a cell when RNA polymerase transcribes the coding region into mRNA, which is then trans-RNA spliced (if the coding region contains introns) and translated into the protein encoded by the coding region.

“Transcriptional and translational control regions” are DNA regulatory regions, such as promoters, enhancers, terminators, and the like, that provide for the expression of a coding region in a host cell. In eukaryotic cells, polyadenylation signals are control regions.

The term “operably associated” refers to the association of nucleic acid sequences on a single nucleic acid fragment so that the function of one is affected by the other. For example, a promoter is operably associated with a coding region when it is capable of affecting the expression of that coding region (i.e., that the coding region is under the transcriptional control of the promoter). Coding regions can be operably associated to regulatory regions in sense or antisense orientation.

The term “expression,” as used herein, refers to the transcription and stable accumulation of sense (mRNA) or antisense RNA derived from the nucleic acid fragment of the invention. Expression may also refer to translation of mRNA into a polypeptide.

### Nitrogen and CBP

Nitrogen composes approximately ten percent of a dry cell mass, the largest element mass fraction after carbon and oxygen. Lignocellulosic biomass is a low nitrogen substrate, and to support microorganism growth, nitrogen must be added to the medium during fermentation. The cost of nitrogen supplementation is a significant factor of the overall medium expense. Nitrogen can be supplied in several forms, including complex additives (proteins), ammonium salts, ammonium hydroxide, ammonia gas, or urea. Complex additives are often prohibitively expensive to serve as a nitrogen source in an industrial medium. Ammonium salts and ammonium hydroxide offer lower cost alternatives, but their use impacts the medium pH—either by decreasing pH upon utilization of ammonium salts, or by increasing the pH upon addition to the media by ammonium hydroxide. To maintain a desirable pH, a neutralizing agent must be used at additional cost. Ammonia gas is a low cost chemical that does not impact pH; however, it is a hazardous chemical that must be stored at high pressure which is undesirable from a process safety standpoint.

Urea offers a low cost, safe nitrogen source that does not require additional pH neutralization when used as a medium additive, and as such, is attractive for an industrial process. However, in order for microorganisms to utilize urea they must have the urease enzyme, which converts urea to ammonium and carbon dioxide. Urease activity is a common but not ubiquitous phenotype of bacteria. Studies have indicated that between 8-20% of cultured microorganisms from human feces and 0-50% of cultured organisms from cow rumens displayed urease activity. See Wozny et al., Appl. Environ. Microbiol. 33:1097-1104 (1977).

The saccharolytic, thermophilic, anaerobic eubacteria, including species belonging to the genera Thermoanaerobacter, Thermoanaerobium, Thermobacterioides, and Clostridium are highly useful for use in consolidated bioprocessing (CBP) systems. Particular species belonging to these genera have certain advantageous functionalities for CBP systems over others. A comparison of T. saccharolyticum with C. thermocellum, as discussed further below, reveals certain characteristics of T. saccharolyticum that are advantageous for CBP.

Comparison of T. saccharolyticum and C. thermocellum

Plant biomass is composed of a heterogeneous matrix whose primary components are cellulose, hemicellulose (xylan), and lignin. Biologically, cellulose and hemicellulose can be degraded by anaerobic metabolism, while lignin requires oxygen to be degraded into more basic components. In thermophilic anaerobic bacteria the fermentation of cellulose and hemicellulose is largely divided among different species, with cellulose fermentation proceeding primarily through cellulolytic organisms such as Clostridium thermocellum or Clostridium straminisolvens, while hemicellulose fermentation is carried out primarily by xylanolytic species of Thermoanaerobacterium, Thermoanaerobacter, or other related genera. Other distinguishing characteristics of these two organism types include the fermentation of monosaccharides, the minimum pH tolerated for growth, and the ability to use urea as a nitrogen source.

Certain distinguishing characteristics of cellulolytic and xylanolytic thermophilic bacteria are shown below in Table 1 and described further in Demain et al., MMBR 69:124-154 (2005) and Lee et al., Intl. J. of Systematic Bacteriology 43:41-51 (1993).

TABLE 1

| | Rapidly Ferments | | | Minimum | |
|---|---|---|---|---|---|
| | Cellulose | Xylan | Monosaccharides | pH | Urease |
| Cellulolytic | Yes | No | No | 6 | Yes |
| thermophilic | | | | | |
| bacteria | | | | | |
| Xylanolytic | No | Yes | Yes | 4-5 | No |
| thermophilic | | | | | |
| bacteria | | | | | |

### Urease

The present invention is directed to the heterologous expression of at least two or three catalytic subunits of urease together with four accessory genes comprising the urease operon in an anaerobic, thermophilic host for use in a consolidated bioprocessing system. The urease enzyme contains an active site with two Ni²⁺ ions, which requires the transport of nickel into the cell, proper capture of nickel by the metallochaperone ureE, and activation of the urease apo-enzyme by ureD, ureF, and ureG. See Remaut et al., J. Biol. Chem. 276:49365-49370 (2001). It would not necessarily be expected that cloning and expression of heterologous urease genes in a Thermoanaerobacterium or Thermoanaerobacter host would lead to an active urease enzyme. Urea-utilizing organisms often contain urea ABC-type transporters, which are not present in Thermoanaerobacterium or Thermoanaerobacter strains. Transport of urea through the cell membrane via passive diffusion without a dedicated transporter occurs at high external urea concentrations (Siewe et al., Archives of Microbiology 169:411-416 (1998)), but passive urea transport at a base rate to support rapid growth would not have necessarily been expected. Finally, the use of urea as a nitrogen source unexpectedly allows for increased ethanol titers compared to the use of nitrogen from complex additives or ammonium salts in T. saccharolyticum strains engineered to produce ethanol at high yield.

In certain embodiments, the invention is directed to an anaerobic thermophilic host, such as a Thermoanaerobacterium or Thermoanaerobacter host capable of utilizing urea by expression of a urease enzyme. In particular embodiments, the urease genes (α, β, γ, D, E, F, G) that are heterologously expressed in a Thermoanaerobacterium or Thermoanaerobacter host are derived from a microorganism that natively expresses the urease enzyme, such as Clostridium thermocellum (C. thermocellum). In further embodiments, the urease genes are under the control of an appropriate promoter, such as the C. thermocellum cbp promoter, or the native C. thermocellum urease promoter as part of a synthetic operon.

### Polynucleotides of the Invention

The present invention provides for the use of urease genes (α, β, γ, D, E, F, G) polynucleotide sequences from anaerobic, thermophilic organisms that natively express the urease enzyme, such as C. thermocellum.

The C. thermocellum urease gene (α, β, γ, D, E, F, G) nucleic acid sequences are available in GenBank (Accession Numbers YP_{—}001038230, YP_{—}001038231, YP_{—}001038232, YP_{—}001038226, YP_{—}001038229, YP_{—}001038228, and YP_{—}001038227, respectively).

The ureα protein sequence is:

(SEQ ID NO: 1)
MSVKISGKDYAGMYGPTKGDRVRLADTDLIIEIEEDYTVYGDECKFGGG
KSIRDGMGQSPSAARDDKVLDLVITNAIIFDTWGIVKGDIGIKDGKIAG
IGKAGNPKVMSGVSEDLIIGASTEVITGEGLIVTPGGIDTHIHFICPQQ
IETALFSGITTMIGGGTGPADGTNATTCTPGAFNIRKMLEAAEDFPVNL
GFLGKGNASFETPLIEQIEAGAIGLKLHEDWGTTPKAIDTCLKVADLFD
VQVAIHTDTLNEAGFVENTIAAIAGRTIHTYHTEGAGGGHAPDIIKIAS
RMNVLPSSTNPTMPFTVNTLDEHLDMLMVCHHLDSKVKEDVAFADSRIR
PETIAAEDILHDMGVFSMMSSDSQAMGRVGEVIIRTWQTAHKMKLQRGA
LPGEKSGCDNIRAKRYLAKYTINPAITHGISQYVGSLEKGKIADLVLWK
PAMFGVKPEMIIKGGFIIAGRMGDANASIPTPQPVIYKNMFGAFGKAKY
GTCVTFVSKASLENGVVEKMGLQRKVLPVQGCRNISKKYMVHNNATPEI
EVDPETYEVKVDGEIITCEPLKVLPMAQRYFLF

The ureα protein is encoded by the following sequence:

(SEQ ID NO: 8)
ATGAGTGTAAAAATAAGCGGCAAAGATTATGCCGGTATGTATGGCCC
GACAAAAGGCGACAGGGTGAGGCTGGCAGACACGGATCTCATTATTG
AGATTGAGGAAGATTACACGGTTTATGGAGATGAGTGCAAATTCGGA
GGAGGTAAATCCATAAGGGACGGAATGGGCCAGTCTCCTTCGGCTGC
AAGAGATGACAAGGTTTTGGATTTGGTAATTACCAATGCCATAATCTT
TGACACATGGGGGATTGTAAAGGGAGATATAGGTATAAAAGACGGAA
AAATAGCCGGAATCGGGAAGGCGGGAAATCCGAAAGTAATGAGCGGC
GTGTCGGAGGATTTAATAATCGGGGCCTCTACCGAAGTTATTACCGGA
GAAGGACTTATTGTGACTCCGGGAGGAATTGATACACATATACATTTT
ATATGCCCCCAGCAGATTGAGACCGCATTGTTCAGCGGTATCACAACA
ATGATTGGTGGCGGAACGGGACCGGCAGACGGAACCAATGCCACCAC
TTGCACACCGGGAGCCTTTAACATCCGGAAAATGTTAGAGGCGGCAG
AGGACTTTCCGGTAAATTTAGGTTTTTTGGGGAAAGGGAATGCTTCTTT
TGAGACTCCTCTGATAGAACAGATTGAAGCAGGGGCGATTGGCTTAAA
GCTCCATGAGGATTGGGGAACCACACCCAAGGCTATAGATACATGCCT
GAAAGTTGCGGATCTTTTTGATGTACAGGTGGCTATACATACCGATAC
ACTGAACGAGGCAGGATTTGTAGAGAATACTATAGCGGCTATAGCCG
GAAGGACAATTCACACTTACCATACCGAGGGAGCGGGCGGCGGGCAC
GCACCGGACATAATTAAAATTGCATCACGCATGAATGTACTGCCCTCG
TCTACCAATCCCACCATGCCTTTTACCGTCAATACATTGGATGAACATC
TCGATATGCTTATGGTATGCCATCATCTTGACAGCAAGGTAAAAGAGG
ACGTTGCTTTTGCCGATTCGAGGATCCGGCCTGAGACAATAGCCGCAG
AAGACATACTGCACGATATGGGAGTATTCAGCATGATGAGTTCCGATT
CCCAGGCCATGGGACGCGTGGGAGAGGTTATTATAAGGACCTGGCAG
ACTGCACATAAAATGAAGCTTCAAAGAGGTGCCCTGCCGGGGGAAAA
GAGCGGCTGTGACAATATAAGGGCTAAAAGATACCTTGCCAAGTATA
CCATAAACCCTGCTATAACCCATGGAATTTCACAGTATGTGGGCTCCC
TGGAGAAAGGGAAAATAGCCGACTTGGTCCTCTGGAAGCCTGCAATG
TTTGGTGTAAAGCCTGAAATGATTATTAAGGGCGGCTTTATAATAGCC
GGCAGGATGGGCGATGCAAATGCGTCCATACCCACACCTCAGCCTGTA
ATATATAAAAACATGTTCGGTGCCTTCGGAAAGGCAAAGTACGGAAC
CTGTGTGACTTTTGTTTCAAAGGCTTCGCTGGAAAATGGCGTTGTGGA
AAAGATGGGGCTTCAAAGAAAAGTGCTTCCGGTCCAGGGATGCAGGA
ATATCTCAAAAAAATATATGGTACACAACAATGCAACGCCTGAAATTG
AAGTTGATCCTGAAACCTATGAGGTAAAGGTGGACGGTGAGATTATCA
CCTGCGAACCATTAAAGGTCTTACCCATGGCGCAGAGATATTTCTTGT
TTTAA.

The ureβ protein sequence is:

(SEQ ID NO: 2)
MIPGEYIIKNEFITLNDGRRTLNIKVSNTGDRPVQVGSHYHFFEVNRYLEF
DRKSAFGMRLDIPSGTAVRFEPGEEKTVQLVEIGGSREIYGLNDLTCGPLD
REDLSNVFKKAKELGFKGVE.

The ureβ protein is encoded by the following sequence:

(SEQ ID NO: 9)
ATGATTCCTGGCGAGTACATTATAAAAAATGAGTTTATCACATTGAAT
GATGGAAGAAGGACTTTAAATATCAAGGTTTCAAATACAGGAGACCG
GCCCGTTCAGGTGGGGTCCCACTACCATTTCTTCGAAGTTAATCGGTAT
CTTGAGTTTGACAGAAAAAGCGCTTTCGGAATGAGACTGGACATTCCT
TCGGGTACTGCGGTAAGGTTTGAGCCGGGGGAGGAAAAGACAGTTCA
ACTGGTTGAAATAGGGGGAAGCAGAGAAATTTACGGACTTAATGATC
TGACTTGCGGTCCCCTTGACAGAGAAGATTTGTCCAATGTGTTTAAAA
AGGCGAAAGAGCTGGGGTTCAAGGGGGTGGAATAA.

The ureγ protein sequence is:

(SEQ ID NO: 3)
MHLTPRETEKLMLHYAGELARKRKERGLKLNYPEAVALISAELMEAARD
GKTVTELMQYGAKILTRDDVMEGVDAMMEIQIEATFPDGTKLVTVHNPI
R.

The ureγ protein is encoded by the following sequence:

(SEQ ID NO: 10)
GTGCATTTGACGCCCAGGGAAACCGAAAAATTGATGCTTCATTATGCC
GGTGAACTGGCAAGAAAACGAAAAGAAAGAGGTCTTAAGCTTAATTA
TCCGGAAGCTGTAGCCCTTATAAGCGCTGAACTGATGGAGGCCGCCCG
GGACGGAAAAACTGTAACGGAACTGATGCAGTATGGAGCAAAGATAC
TGACCAGGGATGATGTAATGGAAGGAGTTGACGCCATGATACATGAA
ATTCAGATAGAGGCAACTTTCCCGGACGGTACAAAGCTTGTTACCGTT
CACAATCCTATACGCTAG.

The ureD protein sequence is:

(SEQ ID NO: 4)
MKNKFGKESRLYIRAKVSDGKTCLQDSYFTAPFKIAKPFYEGHGGFMNL
MVMSASAGVMEGDNYRIEVELDKGARVKLEGQSYQKIHRMKNGTAVQYN
SFTLADGAFLDYAPNPTIPFADSAFYSNTECRMEEGSAFIYSEILAAGR
VKSGEIFRFREYHSGIKIYYGGELIFLENQFLFPKVQNLEGIGFFEGFT
HQASMGFFCKQISDELIDKLCVMLTAMEDVQFGLSKTKKYGFVVRILGN
SSDRLESILKLIRNILY.

The ureD protein is encoding by the following sequence:

(SEQ ID NO: 11)
ATGAAGAATAAATTCGGAAAAGAAAGCAGGCTGTACATAAGAGCAAA
GGTTTCAGACGGAAAAACATGCCTTCAGGATTCGTATTTCACAGCACC
TTTTAAAATAGCCAAACCCTTTTATGAAGGGCATGGCGGATTTATGAA
TCTTATGGTTATGTCAGCTTCAGCGGGAGTTATGGAGGGTGACAATTA
CAGGATTGAAGTGGAATTGGACAAAGGCGCAAGAGTGAAACTGGAAG
GCCAGTCCTACCAGAAGATTCACCGGATGAAAAATGGAACGGCAGTG
CAGTACAACAGTTTTACCCTTGCAGACGGAGCGTTTTTGGATTATGCTC
CCAACCCCACCATACCTTTTGCCGACTCAGCATTTTATTCAAATACAG
AATGCAGGATGGAAGAAGGCTCAGCCTTTATCTATTCGGAGATACTGG
CCGCGGGCAGGGTTAAGAGCGGTGAAATTTTCCGGTTCAGGGAATATC
ACAGCGGGATAAAGATTTATTACGGCGGGGAACTGATTTTTCTTGAAA
ATCAGTTCCTTTTTCCAAAAGTGCAGAATCTTGAAGGAATCGGATTTTT
TGAAGGTTTTACACATCAGGCGTCAATGGGTTTTTTTTGTAAGCAGAT
AAGCGATGAACTTATTGATAAACTTTGTGTAATGCTTACGGCCATGGA
GGATGTCCAGTTCGGATTGAGCAAAACAAAGAAGTATGGCTTTGTTGT
TCGGATTCTCGGAAACAGCAGTGATAGGCTGGAAAGTATTCTAAAACT
GATTAGAAATATCCTCTATTAG.

The ureE protein sequence is:

(SEQ ID NO: 5)
MIVERVLYNIKDIDLEKLEVDFVDIEWYEVQKKILRKLSSNGIEVGIRN
SNGEALKEGDVLWQEGNKVLVVRIPYCDCIVLKPQNMYEMGKTCYEMGN
RHAPLFIDGDELMTPYDEPLMQALIKCGLSPYKKSCKLTTPLGGNLHGY
SHSHSH.

The ureE protein is encoded by the following sequence:

(SEQ ID NO: 12)
ATGATTGTTGAAAGAGTTTTGTATAATATCAAAGATATCGACTTGGAA
AAATTGGAAGTTGATTTCGTGGATATTGAATGGTATGAAGTTCAAAAA
AAAATACTACGCAAATTAAGTTCCAACGGAATTGAAGTTGGAATAAG
AAACAGCAACGGTGAGGCTTTAAAAGAAGGAGACGTATTGTGGCAGG
AGGGAAATAAAGTTTTGGTTGTAAGGATTCCCTATTGCGACTGTATCG
TGCTGAAGCCTCAAAATATGTATGAGATGGGCAAGACTTGCTATGAGA
TGGGAAACAGACATGCACCTCTTTTTATTGATGGAGATGAGCTGATGA
CTCCCTATGATGAGCCGTTGATGCAGGCATTGATAAAATGCGGGCTTT
CACCTTACAAAAAGAGCTGTAAACTTACAACGCCCTTAGGAGGTAATC
TTCATGGATACTCCCATTCTCATTCCCACTGA.

The ureF protein sequence is:

(SEQ ID NO: 6)
MDTPILIPTDMNRIPFFYLLQISDPLFPIGGFTQSYGLETYVQKGIVHD
AETSKKYLESYLLNSFLYNDLLAVRLSWEYTQKGNLNKVLELSEVFSAS
KAPRELRAANEKLGRRFIKILEFVLGENEMFCEMYEKVGRGSVEVSYPV
MYGFCTNLLNIGKKEALSAVTYSAASSIINNCAKLVPISQNEGQKILFN
AHGIFRRLLERVEELDEEYLGSCCFGFDLRAMQHERLYTRLYIS.

The ureF protein is encoded by the following sequence:

(SEQ ID NO: 13)
ATGGATACTCCCATTCTCATTCCCACTGATATGAATAGAATACCCTTTT
TTTACCTTTTACAGATTAGCGATCCGCTGTTTCCGATAGGAGGTTTTAC
CCAATCCTATGGGCTTGAAACCTATGTGCAAAAAGGGATTGTCCATGA
TGCTGAAACTTCGAAAAAATACCTTGAAAGCTATCTTTTAAACAGCTT
TTTGTACAATGATTTATTGGCCGTCAGGCTTTCCTGGGAATATACCCAA
AAAGGAAATTTGAATAAGGTATTGGAACTTTCGGAAGTTTTTTTCGGCC
TCAAAGGCGCCGAGGGAGCTTAGAGCGGCAAATGAAAAGCTCGGCAG
GAGGTTTATAAAGATACTGGAATTTGTTTTGGGCGAAAACGAAATGTT
TTGCGAAATGTATGAAAAAGTGGGGAGAGGAAGTGTGGAAGTTTCGT
ATCCTGTAATGTACGGTTTTTGTACAAATCTTCTCAATATCGGAAAAA
AGGAAGCGTTGTCGGCGGTTACTTATAGCGCGGCATCTTCCATAATAA
ATAACTGTGCAAAATTGGTACCTATCAGCCAGAACGAAGGGCAGAAG
ATTTTATTCAATGCCCATGGCATTTTCCGAAGGCTTTTGGAAAGAGTG
GAGGAACTGGACGAGGAATATCTGGGAAGCTGCTGCTTTGGATTTGAC
TTAAGAGCCATGCAGCATGAAAGGCTCTATACAAGGCTTTATATATCC
TAG.

The ureG protein sequence is:

(SEQ ID NO: 7)
MNYVKIGVGGPVGSGKTALIEKLTRILADSYSIGVVTNDIYTKEDAEFL
IKNSVLPKERIIGVETGGCPHTAIREDASMNLEAVEELVQRFPDIQIVF
IESGGDNLSATFSPELADATIYVIDVAEGDKIPRKGGPGITRSDLLVIN
KIDLAPYVGASLEVMERDSKKMRGEKPFIFTNLNTNEGVDKIIDWIKKS
VLLEGV.

The ureG protein is encoded by the following sequence:

(SEQ ID NO: 14)
ATGAATTATGTGAAAATCGGCGTGGGAGGTCCGGTAGGATCGGGCAA
GACCGCCCTTATAGAAAAATTGACAAGAATATTGGCTGATTCTTACAG
CATCGGGGTGGTTACCAACGATATATACACAAAAGAGGACGCGGAAT
TTTTAATAAAGAACAGTGTACTTCCCAAAGAGAGGATAATTGGAGTGG
AAACCGGCGGCTGCCCTCATACGGCTATTCGCGAGGATGCTTCCATGA
ACCTTGAAGCTGTGGAGGAACTGGTACAGCGGTTCCCTGATATTCAAA
TTGTGTTTATTGAAAGCGGGGGAGACAATCTTTCCGCAACTTTCAGTC
CGGAACTGGCCGATGCCACCATATATGTCATCGATGTGGCCGAAGGTG
ACAAAATTCCCCGAAAAGGCGGCCCGGGAATAACCCGGTCGGATTTA
CTGGTCATAAATAAAATTGATCTGGCTCCATACGTGGGAGCAAGCCTT
GAGGTAATGGAAAGGGATTCAAAGAAGATGAGGGGTGAGAAACCTTT
TATATTCACCAATTTGAATACAAATGAAGGTGTGGATAAGATTATCGA
TTGGATTAAGAAAAGCGTCCTTTTGGAAGGTGTGTAA.

The present invention also provides for the use of an isolated polynucleotide comprising a nucleic acid at least about 70%, 75%, or 80% identical, at least about 90% to about 95% identical, or at least about 96%, 97%, 98%, 99% or 100% identical to any of SEQ ID NOs: 8-14, or fragments, variants, or derivatives thereof.

The present invention also encompasses the use of variants of the urease gene (α, β, γ, D, E, F, G) genes, as described above. Variants may contain alterations in the coding regions, non-coding regions, or both. Examples are polynucleotide variants containing alterations which produce silent substitutions, additions, or deletions, but do not alter the properties or activities of the encoded polypeptide. In certain embodiments, nucleotide variants are produced by silent substitutions due to the degeneracy of the genetic code. In further embodiments, urease gene (α, β, γ, D, E, F, G) polynucleotide variants can be produced for a variety of reasons, e.g., to optimize codon expression for a particular host (e.g., change codons in the C. thermocellum urease gene (α, β, γ, D, E, F, G) mRNAs to those preferred by a host such as T. saccharolyticum).

Also provided in the present invention are allelic variants, orthologs, and/or species homologs. Procedures known in the art can be used to obtain full-length genes, allelic variants, splice variants, full-length coding portions, orthologs, and/or species homologs of genes corresponding to any of SEQ ID NOs: 8-14, using information from the sequences disclosed herein. For example, allelic variants and/or species homologs may be isolated and identified by making suitable probes or primers from the sequences provided herein and screening a suitable nucleic acid source for allelic variants and/or the desired homologue.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the particular polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of any of SEQ ID NOs: 8-14, or any fragment or domain specified as described herein.

As a practical matter, whether any particular nucleic acid molecule or polypeptide is at least 80%, 85%, 90%, 95%, 96%, 97%, 98% or 99% identical to a nucleotide sequence or polypeptide of the present invention can be determined conventionally using known computer programs. A method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245.) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

Some embodiments of the invention encompass a nucleic acid molecule comprising at least 10, 20, 30, 35, 40, 50, 60, 70, 80, 90, 100, 200, 300, 400, 500, 600, 700, or 800 consecutive nucleotides or more of any of SEQ ID NOs: 8-14, or domains, fragments, variants, or derivatives thereof.

The polynucleotide of the present invention may be in the form of RNA or in the form of DNA, which DNA includes cDNA, genomic DNA, and synthetic DNA. The DNA may be double stranded or single-stranded, and if single stranded may be the coding strand or non-coding (anti-sense) strand. The coding sequence which encodes the mature polypeptide may be identical to the coding sequence encoding SEQ ID NOs: 1-7 or may be a different coding sequence which coding sequence, as a result of the redundancy or degeneracy of the genetic code, encodes the same mature polypeptide as the DNA of any one of SEQ ID NOs: 8-14.

In certain embodiments, the present invention provides an isolated polynucleotide comprising a nucleic acid fragment which encodes at least 10, at least 20, at least 30, at least 40, at least 50, at least 60, at least 70, at least 80, at least 90, at least 95, or at least 100 or more contiguous amino acids of SEQ ID NOs: 1-7.

The polynucleotide encoding for the mature polypeptide of SEQ ID NOs: 1-7 or the mature polypeptide encoded by the deposited clone may include: only the coding sequence for the mature polypeptide; the coding sequence of any domain of the mature polypeptide; and the coding sequence for the mature polypeptide (or domain-encoding sequence) together with non-coding sequence, such as introns or non-coding sequence 5′ and/or 3′ of the coding sequence for the mature polypeptide.

Thus, the term “polynucleotide encoding a polypeptide” encompasses a polynucleotide which includes only sequences encoding for the polypeptide as well as a polynucleotide which includes additional coding and/or non-coding sequences.

In further aspects of the invention, nucleic acid molecules having sequences at least 90%, 95%, 96%, 97%, 98% or 99% identical to the nucleic acid sequences disclosed herein, encode a polypeptide having functional urease gene (α, β, γ, D, E, F, G) activity. By “a polypeptide having urease gene (α, β, γ, D, E, F, G) functional activity” is intended polypeptides exhibiting activity similar, but not necessarily identical, to a functional activity of the urease (α, β, γ, D, E, F, G) polypeptides of the present invention, as measured, for example, in a particular biological assay. For example, a urease gene (α, β, γ, D, E, F, G) functional activity can routinely be measured by determining the ability of the encoded urease enzyme to utilize nitrogen, or by measuring the level of urease activity.

Of course, due to the degeneracy of the genetic code, one of ordinary skill in the art will immediately recognize that a large portion of the nucleic acid molecules having a sequence at least 90%, 95%, 96%, 97%, 98%, or 99% identical to the nucleic acid sequence of any of SEQ ID NOs: 8-14, or fragments thereof, will encode polypeptides “having urease gene (α, β, γ, D, E, F, G) functional activity.” In fact, since degenerate variants of any of these nucleotide sequences all encode the same polypeptide, in many instances, this will be clear to the skilled artisan even without performing the above described comparison assay. It will be further recognized in the art that, for such nucleic acid molecules that are not degenerate variants, a reasonable number will also encode a polypeptide having urease gene (α, β, γ, D, E, F, G) functional activity.

Fragments of the full length gene of the present invention may be used as a hybridization probe for a cDNA library to isolate the full length cDNA and to isolate other cDNAs which have a high sequence similarity to the urease genes (α, β, γ, D, E, F, G) of the present invention, or genes encoding for a protein with similar biological activity. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. Hybridization does occur between imperfectly complementary molecules with the result that a certain fraction of the bases in the hybridized region are not paired with the proper complementary base.

In certain embodiments, a hybridization probe may have at least 30 bases and may contain, for example, 50 or more bases. The probe may also be used to identify a cDNA clone corresponding to a full length transcript and a genomic clone or clones that contain the complete gene including regulatory and promoter regions, exons, and introns. An example of a screen comprises isolating the coding region of the gene by using the known DNA sequence to synthesize an oligonucleotide probe. Labeled oligonucleotides having a sequence complementary to that of the gene of the present invention are used to screen a library of bacterial or fungal cDNA, genomic DNA or mRNA to determine which members of the library the probe hybridizes to.

The present invention further relates to polynucleotides which hybridize to the herein above-described sequences if there is at least 70%, at least 90%, or at least 95% identity between the sequences. The present invention particularly relates to polynucleotides which hybridize under stringent conditions to the hereinabove-described polynucleotides. As herein used, the term “stringent conditions” means hybridization will occur only if there is at least 95% or at least 97% identity between the sequences. In certain aspects of the invention, the polynucleotides which hybridize to the hereinabove described polynucleotides encode polypeptides which either retain substantially the same biological function or activity as the mature polypeptide encoded by the DNAs of any of SEQ ID NOs: 8-14, or the deposited clones.

Alternatively, polynucleotides which hybridize to the hereinabove-described sequences may have at least 20 bases, at least 30 bases, or at least 50 bases which hybridize to a polynucleotide of the present invention and which has an identity thereto, as hereinabove described, and which may or may not retain activity. For example, such polynucleotides may be employed as probes for the polynucleotide of any of SEQ ID NOs: 8-14, or the deposited clones, for example, for recovery of the polynucleotide or as a diagnostic probe or as a PCR primer.

Hybridization methods are well defined and have been described above. Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the sequence.

For example, genes encoding similar proteins or polypeptides to those of the instant invention could be isolated directly by using all or a portion of the instant nucleic acid fragments as DNA hybridization probes to screen libraries from any desired bacteria using methodology well known to those skilled in the art. Specific oligonucleotide probes based upon the instant nucleic acid sequences can be designed and synthesized by methods known in the art (see, e.g., Maniatis, 1989). Moreover, the entire sequences can be used directly to synthesize DNA probes by methods known to the skilled artisan such as random primers DNA labeling, nick translation, or end-labeling techniques, or RNA probes using available in vitro transcription systems.

In certain aspects of the invention, polynucleotides which hybridize to the hereinabove-described sequences having at least 20 bases, at least 30 bases, or at least 50 bases which hybridize to a polynucleotide of the present invention may be employed as PCR primers. Typically, in PCR-type amplification techniques, the primers have different sequences and are not complementary to each other. Depending on the desired test conditions, the sequences of the primers should be designed to provide for both efficient and faithful replication of the target nucleic acid. Methods of PCR primer design are common and well known in the art. Generally two short segments of the instant sequences may be used in polymerase chain reaction (PCR) protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding microbial genes. Alternatively, the second primer sequence may be based upon sequences derived from the cloning vector. For example, the skilled artisan can follow the RACE protocol (Frohman et al., PNAS USA 85:8998 (1988)) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., PNAS USA 86:5673 (1989); Loh et al., Science 243:217 (1989)).

In addition, specific primers can be designed and used to amplify a part of or full-length of the instant sequences. The resulting amplification products can be labeled directly during amplification reactions or labeled after amplification reactions, and used as probes to isolate full length DNA fragments under conditions of appropriate stringency.

Therefore, the nucleic acid sequences and fragments thereof of the present invention may be used to isolate genes encoding homologous proteins from the same or other fungal species or bacterial species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, Mullis et al., U.S. Pat. No. 4,683,202; ligase chain reaction (LCR) (Tabor, S. et al., Proc. Acad. Sci. USA 82, 1074, (1985)); or strand displacement amplification (SDA), Walker, et al., Proc. Natl. Acad. Sci. U.S.A., 89, 392, (1992)).

### Polypeptides of the Invention

The present invention further relates to the expression of an urease enzyme from an anaerobic, thermophilic organism that natively expresses such an enzyme. In particular aspects of the invention, the urease enzyme is composed of C. thermocellum urease gene (α, β, γ, D, E, F, G) polypeptides and is expressed in a host cell, such as a Thermoanaerobacterium or Thermoanaerobacter strain, e.g., T. saccharolyticum. The present invention further encompasses polypeptides which comprise, or alternatively consist of, an amino acid sequence which is at least 80%, 85%, 90%, 95%, 96%, 97%, 98%, 99% identical to, for example, the polypeptide sequence shown in SEQ ID NOs: 1-7, and/or domains, fragments, variants, or derivative thereof, of any of these polypeptides (e.g., those fragments described herein, or domains of any of SEQ ID NOs: 1-7).

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, the amino acid sequences of SEQ ID NOs: 1-7 or to the amino acid sequence encoded by the deposited clones can be determined conventionally using known computer programs. As discussed above, a method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Also as discussed above, manual corrections may be made to the results in certain instances.

In certain aspects of the invention, the polypeptides and polynucleotides of the present invention are provided in an isolated form, e.g., purified to homogeneity.

The present invention also encompasses polypeptides which comprise, or alternatively consist of, an amino acid sequence which is at least 80%, 85%, 90%, 95%, 96%, 97%, 98%, 99% similar to the polypeptide of any of SEQ ID NOs: 1-7, and to portions of such polypeptide with such portion of the polypeptide generally containing at least 30 amino acids and more preferably at least 50 amino acids.

As known in the art “similarity” between two polypeptides is determined by comparing the amino acid sequence and conserved amino acid substitutes thereto of the polypeptide to the sequence of a second polypeptide.

The present invention further relates to a domain, fragment, variant, derivative, or analog of the polypeptide of any of SEQ ID NOs: 1-7.

Fragments or portions of the polypeptides of the present invention may be employed for producing the corresponding full-length polypeptide by peptide synthesis, therefore, the fragments may be employed as intermediates for producing the full-length polypeptides.

Fragments of urease (α, β, γ, D, E, F, G) polypeptides of the present invention encompass domains, proteolytic fragments, deletion fragments and in particular, fragments of C. thermocellum urease (α, β, γ, D, E, F, G) polypeptides which retain any specific biological activity of the urease (α, β, γ, D, E, F, G) protein. Polypeptide fragments further include any portion of the polypeptide which comprises a catalytic activity of the urease enzyme.

The variant, derivative or analog of the polypeptide of any of SEQ ID NOs: 1-7 may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group. Such variants, derivatives and analogs are deemed to be within the scope of those skilled in the art from the teachings herein.

The polypeptides of the present invention further include variants of the polypeptides. A “variant’ of the polypeptide can be a conservative variant, or an allelic variant. As used herein, a conservative variant refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the protein.

By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. Allelic variants, though possessing a slightly different amino acid sequence than those recited above, will still have the same or similar biological functions associated with the C. thermocellum urease enzyme.

The allelic variants, the conservative substitution variants, and members of the urease gene (α, β, γ, D, E, F, G) family, will have an amino acid sequence having at least 75%, at least 80%, at least 90%, at least 95% amino acid sequence identity with a C. thermocellum urease gene (α, β, γ, D, E, F, G) amino acid sequence set forth in any one of SEQ ID NOs: 1-7. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity. N terminal, C terminal or internal extensions, deletions, or insertions into the peptide sequence shall not be construed as affecting homology.

Thus, the proteins and peptides of the present invention include molecules comprising the amino acid sequence of SEQ ID NOs: 1-7 or fragments thereof having a consecutive sequence of at least about 3, 4, 5, 6, 10, 15, 20, 25, 30, 35 or more amino acid residues of the C. thermocellum urease gene (α, β, γ, D, E, F, G) polypeptide sequence; amino acid sequence variants of such sequences wherein at least one amino acid residue has been inserted N- or C terminal to, or within, the disclosed sequence; amino acid sequence variants of the disclosed sequences, or their fragments as defined above, that have been substituted by another residue. Contemplated variants further include those containing predetermined mutations by, e.g., homologous recombination, site-directed or PCR mutagenesis; and derivatives wherein the protein has been covalently modified by substitution, chemical, enzymatic, or other appropriate means with a moiety other than a naturally occurring amino acid (for example, a detectable moiety such as an enzyme or radioisotope).

Using known methods of protein engineering and recombinant DNA technology, variants may be generated to improve or alter the characteristics of the urease polypeptides. For instance, one or more amino acids can be deleted from the N-terminus or C-terminus of the secreted protein without substantial loss of biological function.

Thus, the invention further includes C. thermocellum urease gene (α, β, γ, D, E, F, G) polypeptide variants which show substantial biological activity. Such variants include deletions, insertions, inversions, repeats, and substitutions selected according to general rules known in the art so as have little effect on activity.

The skilled artisan is fully aware of amino acid substitutions that are either less likely or not likely to significantly effect protein function (e.g., replacing one aliphatic amino acid with a second aliphatic amino acid), as further described below.

For example, guidance concerning how to make phenotypically silent amino acid substitutions is provided in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are often surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

The terms “derivative” and “analog” refer to a polypeptide differing from the C. thermocellum urease gene (α, β, γ, D, E, F, G) polypeptides, but retaining essential properties thereof. Generally, derivatives and analogs are overall closely similar, and, in many regions, identical to the C. thermocellum urease gene (α, β, γ, D, E, F, G) polypeptides. The term “derivative” and “analog” when referring to C. thermocellum urease gene (α, β, γ, D, E, F, G) polypeptides of the present invention include any polypeptides which retain at least some of the activity of the corresponding native polypeptide, e.g., the hydrolysis of urea to CO₂ and ammonia.

Derivatives of C. thermocellum urease gene (α, β, γ, D, E, F, G) polypeptides of the present invention, are polypeptides which have been altered so as to exhibit additional features not found on the native polypeptide. Derivatives can be covalently modified by substitution, chemical, enzymatic, or other appropriate means with a moiety other than a naturally occurring amino acid (for example, a detectable moiety such as an enzyme or radioisotope). Examples of derivatives include fusion proteins.

An analog is another form of C. thermocellum urease gene (α, β, γ, D, E, F, G) polypeptides of the present invention. An “analog” also retains substantially the same biological function or activity as the polypeptide of interest, i.e., functions as a component of an enzyme that hydrolyzes urea to CO₂ and ammonia. An analog includes a proprotein which can be activated by cleavage of the proprotein portion to produce an active mature polypeptide.

The polypeptide of the present invention may be a recombinant polypeptide, a natural polypeptide or a synthetic polypeptide, preferably a recombinant polypeptide.

### Heterologous Expression of C. Thermocellum Urease Gene (α, β, γ, D, E, F, G) Polypeptides in Host Cells

In order to address the limitations of the previous systems, the present invention provides C. thermocellum urease gene (α, β, γ, D, E, F, G) polypeptides, or domains, variants, or derivatives thereof that can be effectively and efficiently expressed in a consolidated bioprocessing system.

In certain embodiments of the present invention, a host cell comprising a vector which expresses the urease enzyme encoded by C. thermocellum urease genes (α, β, γ, D, E, F, G) is utilized for consolidated bioprocessing and is optionally co-cultured with additional host cells capable of utilizing urea. For example, the host cell can be an anaerobic, thermophilic host, such as T. saccharolyticum, and the additional host cell can be a different anaerobic, thermophilic host, such as C. thermocellum expressing native urease.

The transformed host cells or cell cultures, as described above, are measured for urease protein content. Protein content can be determined by analyzing the host cell supernatants. In certain embodiments, the high molecular weight material is recovered from the yeast cell supernatant either by acetone precipitation or by buffering the samples with disposable de-salting cartridges. The analysis methods include the traditional Lowry method or protein assay method according to BioRad's manufacturer's protocol. Using these methods, the protein content of saccharolytic enzymes can be estimated.

The transformed host cells or cell cultures, as described above, can be further analyzed for hydrolysis of urea (e.g., by measuring carbon dioxide and ammonia levels).

It will be appreciated that suitable lignocellulosic material can be any feedstock that contains soluble and/or insoluble cellulose, where the insoluble cellulose can be in a crystalline or non-crystalline form. In various embodiments, the lignocellulosic biomass comprises, for example, wood, corn, corn cobs, corn stover, corn fiber, sawdust, bark, leaves, agricultural and forestry residues, grasses such as switchgrass, cord grass, rye grass or reed canary grass, miscanthus, ruminant digestion products, municipal wastes, paper mill effluent, newspaper, cardboard, miscanthus, sugar-processing residues, sugarcane bagasse, agricultural wastes, rice straw, rice hulls, barley straw, cereal straw, wheat straw, canola straw, oat straw, oat hulls, stover, soybean stover, forestry wastes, recycled wood pulp fiber, paper sludge, sawdust, hardwood, softwood or combinations thereof.

### Vectors and Host Cells

The present invention also relates to vectors which include polynucleotides of the present invention, host cells which are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; and yeast plasmids. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively associated with an appropriate expression control sequence(s) (promoter) to direct mRNA synthesis. Representative examples of such promoters include the E. coli, lac or tip, and other promoters known to control expression of genes in prokaryotic or lower eukaryotic cells, the cbp promoter of C. thermocellum, or other promoters for gene expression in anaerobic, thermophilic organisms. The C. thermocellum cbp promoter can have the following sequence:

(SEQ ID NO: 17)
gagtcgtgactaagaacgtcaaagtaattaacaatacagctatttttctcatgcttttacccctttcataaaatttaattttatc
gttatcataaaaaattatagacgttatattgcttgccgggatatagtgctgggcattcgttggtgcaaaatgttcggagta
aggtggatattgatttgcatgttgatctattgcattgaaatgattagttatccgtaaatattaattaatcatatcataaattaatt
atatcataattgttttgacgaatgaaggtttttggataaattatcaagtaaaggaacgctaaaaattttggcgtaaaatatc
aaaatgaccacttgaattaatatggtaaagtagatataatattttggtaaacatgccttcagcaaggttagattagctgttt
ccgtataaattaaccgtatggtaaaacggcagtcagaaaaataagtcataagattccgttatgaaaatatacttcggtag
ttaataataagagatatgaggtaagagatacaagataagagatataaggtacgaatgtataagatggtgcttttaggca
cactaaataaaaaacaaataaacgaaaattttaaggaggacgaaag.

The expression vector also contains a ribosome binding site for translation initiation and a transcription terminator. The vector may also include appropriate sequences for amplifying expression, or may include additional regulatory regions.

In addition, the expression vectors may contain one or more selectable marker genes to provide a phenotypic trait for selection of transformed host cells such as the aph3 gene from the S. facealis plasmid pKD102 conferring thermostable kanamycin resistance (Mai et al, FEMS Microbio. Let. 148:163-167 (1997)).

The vector containing the appropriate DNA sequence as herein, as well as an appropriate promoter or control sequence, may be employed to transform an appropriate host to permit the host to express the protein.

Thus, in certain aspects, the present invention relates to host cells containing the above-described constructs. The host cell can be an anaerobic thermophilic host, such as a Thermoanaerobacterium or Thermoanaerobacter host. A representative example of such a host is T. saccharolyticum. The selection of an appropriate host is deemed to be within the scope of those skilled in the art from the teachings herein.

Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus,Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus. Some examples of thermophilic microorganisms (including bacteria, prokaryotic microorganism, and fungi), which may be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Thermoanaerobacterium thermosaccarolyticum, Thermoanaerobacterium saccharolyticum, Thermobacteroides acetoethylicus, Thermoanaerobium brockii, Methanobacterium thermoautotrophicum, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus Havas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrflcans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, variants thereof, and/or progeny thereof.

In certain embodiments, the present invention relates to thermophilic bacteria of the genera Thermoanaerobacterium or Thermoanaerobacter, including, but not limited to, species selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In certain embodiments, the present invention relates to microorganisms of the genera Geobacillus, Saccharococcus, Paenibacillus, Bacillus, and Anoxybacillus, including, but not limited to, species selected from the group consisting of: Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and progeny thereof.

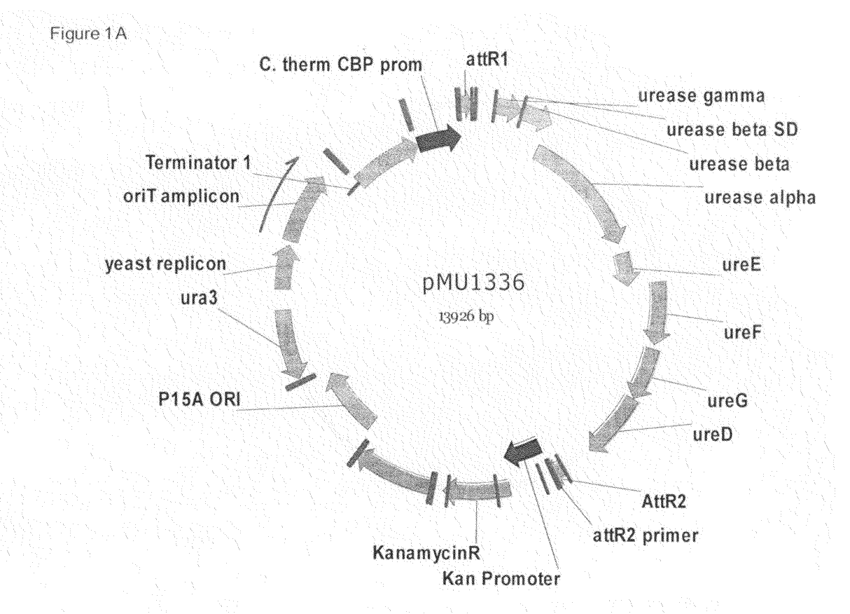

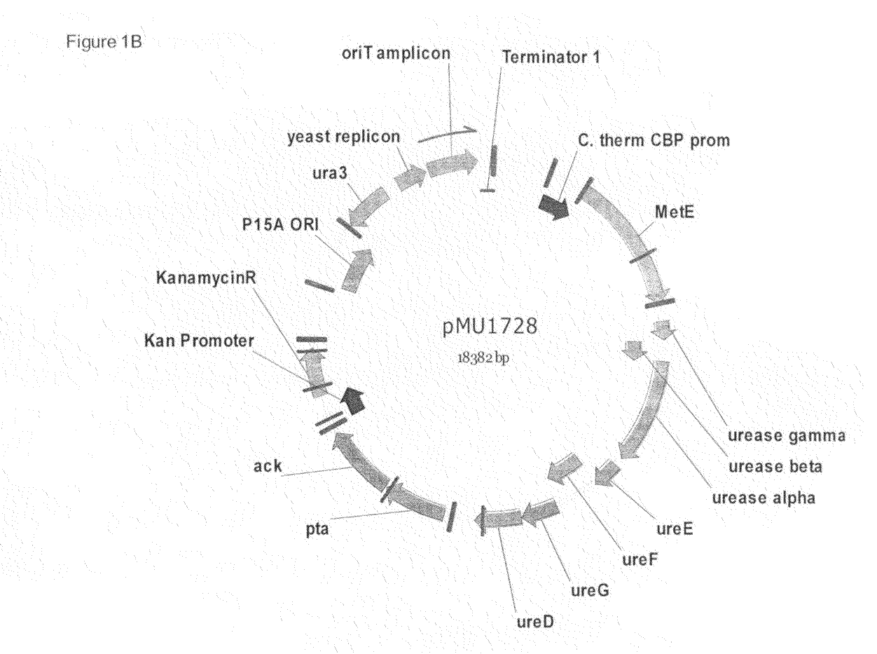

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In one aspect of this embodiment, the construct further comprises regulatory sequences, including, for example, a promoter, operably associated to the sequence. Large numbers of suitable vectors and promoters are known to those of skill in the art, and are commercially available. Two examples of vectors of the present application include pDest-Ct-Urease (pMU1336) and pMetE urease fixA (pMU1728) (as shown in FIGS. 1A and B).

Promoter regions can be selected from any desired gene. Particular named bacterial promoters include lad, lacZ, T3, T7, gpt, lambda PR, PL and trp. Other promoters include those that regulate gene expression in anaerobic, thermophilic organisms, such as the cbp promoter from C. thermocellum. Selection of the appropriate vector and promoter is well within the level of ordinary skill in the art.

Introduction of the construct in other host cells can be effected by calcium phosphate transfection, DEAE-Dextran mediated transfection, or electroporation. (Davis, L., et al., Basic Methods in Molecular Biology, (1986)).

The constructs in host cells can be used in a conventional manner to produce the gene product encoded by the recombinant sequence. Alternatively, the polypeptides of the invention can be synthetically produced by conventional peptide synthesizers.

Following creation of a suitable host cell and growth of the host cell to an appropriate cell density, the selected promoter is induced by appropriate means (e.g., temperature shift or chemical induction) and cells are cultured for an additional period.

The host cell can be cultured in a medium having a particular pH. For example, the host cell can be cultured in medium having a pH range from about 4 to about 9, from about 5 to about 8, or from about 6 to about 8. The host cell can also be cultured in medium having a pH range from about 5 to about 7, from about 6 to about 7, or from about 6.2 to about 6.8.

The host cell can also be cultured in presence of a particular concentration of urea. For example, the concentration of urea can be at least about 0.5 g/L, at least about 1.0 g/L, at least about 1.5 g/L, at least about 2.0 g/L, at least about 2.5 g/L, at least about 3.0 g/L, at least about 3.5 g/L, at least about 4.0 g/L, at least about 4.5 g/L, or at least about 5.0 g/L.

### EXAMPLES

### Example 1

### Heterologous Cloning of Urease Operon into T. saccharolyticum

To create a T. saccharolyticum strain that can utilize urea, the urease genes (α, β, γ, D, E, F, G) (SEQ ID NO: 8 through SEQ ID NO: 14, respectively) from Clostridium thermocellum were heterologously cloned into the genome of T. saccharolyticum under the control of the C. thermocellum cbp promoter (SEQ ID NO:17). These urease genes include the catalytic subunits of the urease enzyme (typically three ureαβγ subunits, but in some species only two subunits) and the accessory proteins ureDEFG that facilitate protein folding and nickel activation.

Two experimental plasmids were created using standard molecular cloning procedures. Schematics of the two plasmids are shown in FIGS. 1A and 1B. pDest-Ct-urease (pMU1336) (FIG. 1A, SEQ ID NO: 15) uses the cbp promoter to directly drive expression of the urease operon, while pMetE_fix_A (pMU1728) (FIG. 1B, SEQ ID NO: 16) has the urease operon downstream of the MetE gene in a synthetic operon under the control of the cbp promoter. A linear PCR product homologous to the 3′ end of the urease operon and the region downstream of orf796 were used for negative selection against the pta/ack locus in pMetE_fix_A plasmid (pMU1728).

The sequence of pDest-Ct-urease (pMU1336) is

(SEQ ID NO: 15)
tggagtttgtaatggatgtggccgactatttttacgttatggataaaggccgcatagtaatggagggaaaaacggaggg
aatcgatcctcatgaaatacaggaaaagattgctatttgataagtatgtcattgataaatatgccataaaattttgcgcctgtaaatttc
gttgttaaaaatattacaaaaaaccaaaagcaatgaataagtatttttagacagggaaaataaattttcctttggttatgccaatttatg
gattaatcaatttaaaagaaggtggtaagagtgcatttgacgcccagggaaaccgaaaaattgatgcttcattatgccggtgaact
ggcaagaaaacgaaaagaaagaggtcttaagcttaattatccggaagctgtagcccttataagcgctgaactgatggaggccgc
ccgggacggaaaaactgtaacggaactgatgcagtatggagcaaagatactgaccagggatgatgtaatggaaggagttgacg
ccatgatacatgaaattcagatagaggcaactttcccggacggtacaaagcttgttaccgttcacaatcctatacgctagagggag
gaaggatgtatgattcctggcgagtacattataaaaaatgagtttatcacattgaatgatggaagaaggactttaaatatcaaggttt
caaatacaggagaccggcccgttcaggtggggtcccactaccatttcttcgaagttaatcggtatcttgagtttgacagaaaaagc
gctttcggaatgagactggacattccttcgggtactgcggtaaggtttgagccgggggaggaaaagacagttcaactggttgaaa
tagggggaagcagagaaatttacggacttaatgatctgacttgcggtccccttgacagagaagatttgtccaatgtgtttaaaaag
gcgaaagagctggggttcaagggggtggaataacatgagtgtaaaaataagcggcaaagattatgccggtatgtatggcccga
caaaaggcgacagggtgaggctggcagacacggatctcattattgagattgaggaagattacacggtttatggagatgagtgca
aattcggaggaggtaaatccataagggacggaatgggccagtctccttcggctgcaagagatgacaaggttttggatttggtaatt
accaatgccataatctttgacacatgggggattgtaaagggagatataggtataaaagacggaaaaatagccggaatcgggaag
gcgggaaatccgaaagtaatgagcggcgtgtcggaggatttaataatcggggcctctaccgaagttattaccggagaaggactt
attgtgactccgggaggaattgatacacatatacattttatatgcccccagcagattgagaccgcattgttcagcggtatcacaaca
atgattggtggcggaacgggaccggcagacggaaccaatgccaccacttgcacaccgggagcctttaacatccggaaaatgtt
agaggcggcagaggactttccggtaaatttaggttttttggggaaagggaatgcttcttttgagactcctctgatagaacagattga
agcaggggcgattggcttaaagctccatgaggattggggaaccacacccaaggctatagatacatgcctgaaagttgcggatct
ttttgatgtacaggtggctatacataccgatacactgaacgaggcaggatttgtagagaatactatagcggctatagccggaagga
caattcacacttaccataccgagggagcgggcggcgggcacgcaccggacataattaaaattgcatcacgcatgaatgtactgc
cctcgtctaccaatcccaccatgccttttaccgtcaatacattggatgaacatctcgatatgcttatggtatgccatcatcttgacagc
aaggtaaaagaggacgttgcttttgccgattcgaggatccggcctgagacaatagccgcagaagacatactgcacgatatggga
gtattcagcatgatgagttccgattcccaggccatgggacgcgtgggagaggttattataaggacctggcagactgcacataaaa
tgaagcttcaaagaggtgccctgccgggggaaaagagcggctgtgacaatataagggctaaaagataccttgccaagtatacc
ataaaccctgctataacccatggaatttcacagtatgtgggctccctggagaaagggaaaatagccgacttggtcctctggaagc
ctgcaatgtttggtgtaaagcctgaaatgattattaagggcggctttataatagccggcaggatgggcgatgcaaatgcgtccata
cccacacctcagcctgtaatatataaaaacatgttcggtgccttcggaaaggcaaagtacggaacctgtgtgacttttgtttcaaag
gcttcgctggaaaatggcgttgtggaaaagatggggcttcaaagaaaagtgcttccggtccagggatgcaggaatatctcaaaa
aaatatatggtacacaacaatgcaacgcctgaaattgaagttgatcctgaaacctatgaggtaaaggtggacggtgagattatcac
ctgcgaaccattaaaggtcttacccatggcgcagagatatttcttgttttaaactgccggaaggttagtttctctgtaaaaaatttatgg
taattgacatttcaaaaaacaattttaaactaaagaaatttttaaataaagaataattttgggaggacttaaaaaaaactcaaaaacata
agttgggtgagatgaaatgattgttgaaagagttttgtataatatcaaagatatcgacttggaaaaattggaagttgatttcgtggata
ttgaatggtatgaagttcaaaaaaaaatactacgcaaattaagttccaacggaattgaagttggaataagaaacagcaacggtgag
gctttaaaagaaggagacgtattgtggcaggagggaaataaagttttggttgtaaggattccctattgcgactgtatcgtgctgaag
cctcaaaatatgtatgagatgggcaagacttgctatgagatgggaaacagacatgcacctctttttattgatggagatgagctgatg
actccctatgatgagccgttgatgcaggcattgataaaatgcgggctttcaccttacaaaaagagctgtaaacttacaacgccctta
ggaggtaatcttcatggatactcccattctcattcccactgatatgaatagaataccctttttttaccttttacagattagcgatccgctg
tttccgataggaggttttacccaatcctatgggcttgaaacctatgtgcaaaaagggattgtccatgatgctgaaacttcgaaaaaat
accttgaaagctatcttttaaacagctttttgtacaatgatttattggccgtcaggctttcctgggaatatacccaaaaaggaaatttga
ataaggtattggaactttcggaagttttttcggcctcaaaggcgccgagggagcttagagcggcaaatgaaaagctcggcagga
ggtttataaagatactggaatttgttttgggcgaaaacgaaatgttttgcgaaatgtatgaaaaagtggggagaggaagtgtggaa
gtttcgtatcctgtaatgtacggtttttgtacaaatcttctcaatatcggaaaaaaggaagcgttgtcggcggttacttatagcgcggc
atcttccataataaataactgtgcaaaattggtacctatcagccagaacgaagggcagaagattttattcaatgcccatggcattttc
cgaaggcttttggaaagagtggaggaactggacgaggaatatctgggaagctgctgctttggatttgacttaagagccatgcagc
atgaaaggctctatacaaggctttatatatcctagtgttaataatcctgtactacattgttatttatcttcttaaggaaggtggagcttatg
aattatgtgaaaatcggcgtgggaggtccggtaggatcgggcaagaccgcccttatagaaaaattgacaagaatattggctgatt
cttacagcatcggggtggttaccaacgatatatacacaaaagaggacgcggaatttttaataaagaacagtgtacttcccaaagag
aggataattggagtggaaaccggcggctgccctcatacggctattcgcgaggatgcttccatgaaccttgaagctgtggaggaa
ctggtacagcggttccctgatattcaaattgtgtttattgaaagcgggggagacaatctttccgcaactttcagtccggaactggcc
gatgccaccatatatgtcatcgatgtggccgaaggtgacaaaattccccgaaaaggcggcccgggaataacccggtcggattta
ctggtcataaataaaattgatctggctccatacgtgggagcaagccttgaggtaatggaaagggattcaaagaagatgaggggtg
agaaaccttttatattcaccaatttgaatacaaatgaaggtgtggataagattatcgattggattaagaaaagcgtccttttggaaggt
gtgtaaattatgaagaataaattcggaaaagaaagcaggctgtacataagagcaaaggtttcagacggaaaaacatgccttcagg
attcgtatttcacagcaccttttaaaatagccaaacccttttatgaagggcatggcggatttatgaatcttatggttatgtcagcttcag
cgggagttatggagggtgacaattacaggattgaagtggaattggacaaaggcgcaagagtgaaactggaaggccagtcctac
cagaagattcaccggatgaaaaatggaacggcagtgcagtacaacagttttacccttgcagacggagcgtttttggattatgctcc
caaccccaccataccttttgccgactcagcattttattcaaatacagaatgcaggatggaagaaggctcagcctttatctattcgga
gatactggccgcgggcagggttaagagcggtgaaattttccggttcagggaatatcacagcgggataaagatttattacggcgg
ggaactgatttttcttgaaaatcagttcctttttccaaaagtgcagaatcttgaaggaatcggattttttgaaggttttacacatcaggc
gtcaatgggttUttttgtaagcagataagcgatgaacttattgataaactttgtgtaatgcttacggccatggaggatgtccagttcg
gattgagcaaaacaaagaagtatggctttgttgttcggattcteggaaacagcagtgataggctggaaagtattctaaaactgatta
gaaatatcctctattagtaaaaataaacactatttttggttatgaaaatcagaactaaatgtattggcagtataaaactgtaaaaacgg
tttaaaaaaagaaagtgtacaagcattgaaaaatatcaacgttaaaaaagttgtaatttagagatgagccggttgttgaaaagttgaa
tgcccaaatcccgttaagttatatcttaatcggaaaaaagaataaaagaaattcgatttatgataaaataccttgacaattttggattac
agctgtaagatataattagacttacaattgtaatctaaaatggaggggcaattatgaaagcagagtctcaaatcacagaagcggaa
ctggaagttatgaaaattctttgggagtatggaaaggccaccagttctcagatcatagtgactggatatgttgtgttttacagtattatg
tagtctgttttttatgcaaaatctaatttaatatattgatatttatatcattttacgtttctcgttcagctttcttgtacaaagtggtaaaccca
gcgaaccatttgaggtgataggtaagattataccgaggtatgasaacgagaattggacctttacagaattactctatgaagcgcca
tatttaaaaagctaccaagacgaagaggatgaagaggatgaggaggcagattgccttgaatatattgacaatactgataagataat
atatcttttatatagaagatatcgccgtatgtaaggatttcagggggcaaggcataggcagcgcgcttatcaatatatctatagaatg
ggcaaagcataaaaacttgcatggactaatgcttgaaacccaggacaataaccttatagcttgtaaattctatcataattgtggtttca
aaatcggctccgtcgatactatgttatacgccaactttcaaaacaactttgaaaaagctgttttctggtatttaaggttttagaatgcaa
ggaacagtgaattggagttcgtcttgttataattagcttcttggggtatctttaaatactgtagaaaagaggaaggaaataataaatg
gctaaaatgagaatatcaccggaattgaaaaaactgatcgaaaaataccgctgcgtaaaagatacggaaggaatgtctcctgcta
aggtatataagctggtgggagaaaatgaaaacctatatttaaaaatgacggacagccggtataaagggaccacctatgatgtgga
acgggaaaaggacatgatgctatggctggaaggaaagctgcctgttccaaaggtcctgcactttgaacggcatgatggctggag
caatctgctcatgagtgaggccgatggcgtcctttgctcggaagagtatgaagatgaacaaagccctgaaaagattatcgagctg
tatgcggagtgcatcaggctctttcactccatcgacatatcggattgtccctatacgaatagcttagacagccgcttagccgaattg
gattacttactgaataacgatctggccgatgtggattgcgaaaactgggaagaagacactccatttaaagatccgcgcgagctgta
tgattttttaaagacggaaaagcccgaagaggaacttgtcttttcccacggcgacctgggagacagcaacatctttgtgaaagatg
gcaaagtaagtggctttattgatcttgggagaagcggcagggcggacaagtggtatgacattgccttctgcgtccggtcgatcag
ggaggatatcggggaagaacagtatgtcgagctattttttgacttactggggatcaagcctgattgggagaaaataaaatattatatt
ttactggatgaattgttttagtacctagatttagatgtctaaaaagctttttagacatctaatcttttctgaagtacatccgcaactgtccat
actctgatgttttatatcttttctaaaagttcgctagataggggtcccgagcgcctacgaggaatttgtatcggatccgcaagagatta
tatcgagtgcctttaagaaggctaaaaattacgaagatgtgatacacaaaaaggcaaaagattacggcaaaaacataccggatag
tcaagttaaaggagtattgaaacagatagagattactgccttaaaccatgtagacaagattgtcgctgctgaaaagacgatgcaga
tagattccctcgtgaagaaaaatatgtcttatgatatgatggatgcattgcaggatatagagaaggatttgataaatcagcagatgtt
ctacaacgaaaatctaataaacataaccaatccgtatgtgaggcagatattcactcagatgagggatgatgagatgcgatttatcac
tatcatacagcagaacatagaatcgttaaagtcaaagccgactgagcccaacagcatagtatatacgacgccgagggaaaataa
atgaaagtagctattataggagcaggctcggcaggcttaactgcagctataaggcttgaatcttatgggataaagcctgatatattt
gagagaaaatcgaaagtcggcgatgcttttaaccatgtaggaggacttttaaatgtcataaataggccaataaatgatcctttagag
tatctaaaaaataactttgatgtagctattgcaccgcttaacaacatagacaagattgtgatgcatgggccaacagtcactcgcaca
attaaaggcagaaggcttggatactttatgctgaaagggcaaggagaattgtcagtagaaagccaactatacaagaaattaaaga
caaatgtcaattttgatgtccacgcagactacaagaacctaaaggaaatttatgattatgtcattgtagcaactggaaatcatcagat
accaaatgagttaggatgttggcagacgcttgttgatacgaggcttaaaattgctgaggtaatcggtaaattcgacccgtctatcag
ctgtccctcctgttcagctactgacggggtggtgcgtaacggcaaaagcaccgccggacatcagcgctagcggagtgtatactg
gcttactatgttggcactgatgagggtgtcagtgaagtgcttcatgtggcaggagaaaaaaggctgcaccggtgcgtcagcaga
atatgtgatacaggatatattccgcttcctcgctcactgactcgctacgctcggtcgttcgactgcggcgagcggaaatggcttacg
aacggggcggagatttcctggaagatgccaggaagatacttaacagggaagtgagagggccgcggcaaagccgtttttccata
ggctccgcccccctgacaagcatcacgaaatctgacgctcaaatcagtggtggcgaaacccgacaggactataaagataccag
gcgtttccccctggcggctccctcgtgcgctctcctgttcctgcctttcggtttaccggtgtcattccgctgttatggccgcgtttgtct
cattccacgcctgacactcagttccgggtaggcagttcgctccaagctggactgtatgcacgaaccccccgttcagtccgaccgc
tgcgcatatccggtaactatcgtcttgagtccaacccggaaagacatgcaaaagcaccactggcagcagccactggtaattgatt
tagaggagttagtcttgaagtcatgcgccggttaaggctaaactgaaaggacaagttttggtgactgcgctcctccaagccagtta
cctcggttcaaagagttggtagctcagagaaccttcgaaaaaccgccctgcaaggcggttttttcgttttcagagcaagagattac
gcgcagaccaaaacgatctcaagaagatcatcttattaatcagataaaatatttctagatttcagtgcaatttatctcttcaaatgtagc
acctgaagtcagccccatacgatataagttgtaattctcatgtttgacagcttatcatcgataagctttaatgcggtagtttatcacagt
taaattgctaacgcagtcaggcacctatacatgcatttacttataatacagtatttagttttgctggccgcatcttctcaaatatgcttcc
cagcctgcttttctgtaacgttcaccctctaccttagcatcccttccctttgcaaatagtcctcttccaacaataataatgtcagatcctg
tagagaccacatcatccacggttctatactgttgacccaatgcgtctcccttgtcatctaaacccacaccgggtgtcataatcaacc
aatcgtaaccttcatctcttccacccatgtctctttgagcaataaagccgataacaaaatctttgtcgctcttcgcaatgtcaacagtac
ccttagtatattctccagtagatagggagcccttgcatgacaattctgctaacatcaaaaggcctctaggttcctttgttacttcttctg
ccgcctgcttcaaaccgctaacaatacctgggcccaccacaccgtgtgcattcgtaatgtctgcccattctgctattctgtatacacc
cgcagagtactgcaatttgactgtattaccaatgtcagcaaattttctgtcttcgaagagtaaaaaattgtacttggcggataatgcct
ttagcggcttaactgtgccctccatggaaaaatcagtcaagatatccacatgtgtttttagtaaacaaattttgggacctaatgcttca
actaactccagtaattccttggtggtacgaacatccaatgaagcacacaagtttgtttgcttttcgtgcatgatattaaatagcttggca
gcaacaggactaggatgagtagcagcacgttccttatatgtagctttcgacatgatttatcttcgtttcctgcaggtttttgttctgtgca
gttgggttaagaatactgggcaatttcatgtttcttcaacactacatatgcgtatatataccaatctaagtctgtgctccttccttcgttct
tccttctgttcggagattaccgaatcaaaaaaatttcaaagaaaccgaaatcaaaaaaaagaataaaaaaaaaatgatgaattgaat
tgaaaagctagcttatcgatgggtccttttcatcacgtgctataaaaataattataatttaaattttttaatataaatatataaattaaaaat
agaaagtaaaaaaagaaattaaagaaaaaatagtttttgttttccgaagatgtaaaagactctagggggatcgccaacaaatacta
catttatcttgctcttcctgctctcaggtattaatgccgaattgtttcatcttgtctgtgtagaagaccacacacgaaaatcctgtgattt
tacatatacttatcgttaatcgaatgtatatctatttaatctgcttttcttgtctaataaatatatatgtaaagtacgctttttgttgaaatattt
aaacctttgtttatttttttttcttcattccgtaactcttctaccttctttatttactttctaaaatccaaatacaaaacataaaaataaataaac
acagagtaaattcccaaattattccatcattaaaagatacgaggcgcgtgtaagttacaggcaagcgatctctaagaaaccattatt
atcatgacattaacctataaaaaaggcctctcgagctagagtcgatcttcgccagcagggcgaggatcgtggcatcaccgaacc
gcgccgtgcgcgggtcgtcggtgagccagagtttcagcaggccgcccaggcggcccaggtcgccattgatgcgggccagct
cgcggacgtgctcatagtccacgacgcccgtgattttgtagccctggccgacggccagcaggtaggccgacaggctcatgccg
gccgccgccgccttttcctcaatcgctatcgttcgtctggaaggcagtacaccttgataggtgggctgcccttcctggttggcttg
gtttcatcagccatccgcttgccctcatctgttacgccggcggtagccggccagcctcgcagagcaggattcccgttgagcaccg
ccaggtgcgaataagggacagtgaagaaggaacacccgctcgcgggtgggcctacttcacctatcctgcccggctgacgccg
ttggatacaccaaggaaagtctacacgaaccattggcaaaatcctgtatatcgtgcgaaaaaggatggatataccgaaaaaatc
gctataatgaccccgaagcagggttatgcagcggaaaagcgctgcttccctgctgttttgtggaatatctaccgactggaaacag
gcaaatgcaggaaattactgaactgaggggacaggcgagagacgatgccaaagagctacaccgacgagctggccgagtggg
ttgaatcccgcgcggccaagaagcgccggcgtgatgaggctgcggttgcgttcctggcggtgagggcggatgtcgatatgcgt
aaggagaaaataccgcatcaggcgcatatttgaatgtatttagaaaaataaacaaaaagagtttgtagaaacgcaaaaaggccat
ccgtcaggatggccttctgataatttgatgcctggcagtttatggcgggcgtcctgcccgccaccctccgggccgttgcttcgca
acgttcaaatccgctcccggcggatttgtcctactcaggagagcgttcaccgacaaacaacagataaaacgaaaggcccagtctt
tcgactgagcctttcgttttatttgatgcctggctcatcgaggtatccaagcgattcaatagtaacagtccttgtatgccctattctttat
cacgatatccatctgcaatagataggtatattcttccggaactgcgtctacttttctttaaatacacattaaactcccccaataaaattca
atataactatattataccacaatccataataatccgcaaccaaaatatgacaaaaatttaaaaaaattttacccaaaatcgttagtaaa
attgctggttccgggttacgctacataaaattttgctgcaaaactagggtaaaaaaaatacaaaccatgcgtcaatagaaattgacg
gcagtatattaaagcagtataatgaatatatggaaaaacaaaagggcaatataatattaaaagggaaatataaacctgaatataag
gaaaagttgcttaatttagccaaattttttactgataatggctttgacctactgaacatgcattgaatgaaatacttgggaaaacagctt
ctggaagattgccagatgacaaacagatgttattggatgtattacaaaatggtgaaaattatattgaacctaatggcaatatagtcag
gtataaaaatggcatatcaatacatatcgataaagaacatggctggataattactataactccaaggaaacgaatagtaaaggaat
ggaggcgaattaatgagtaatgtcgcaatgcaattaatagaaatttgtcggaaatatgtaaataataatttaaacataaatgaatttat
cgaagactttcaagtgctttatgaacaaaagcaagatttattgacagatgaagaaatgagcttgtttgatgatatttatatggcttgtga
atactatgaacaggatgaaaatataagaaatgaatatcacttgtatattggagaaaatgaattaagacaaaaagtgcaaaaacttgt
aaaaaagttagcagcataataaaccgctaaggcatgatagctaaaggagtcgtgactaagaacgtcaaagtaattaacaatacag
ctattatctcatgcttttacccctttcataaaatttaattttatcgttatcataaaaaattatagacgttatattgcttgccgggatatagtgc
tgggcattcgttggtgcaaaatgttaggagtaaggtggatattgatttgcatgttgatctattgcattgaaatgattagttatccgtaaat
attaattaatcatatcataaattaattatatcataattgttttgacgaatgaaggtttttggataaattatcaagtaaaggaacgctaaaaa
ttttggcgtaaaatatcaaaatgaccacttgaattaatatggtaaagtagatataatattttggtaaacatgccttcagcaaggttagat
tagctgtttccgtataaattaaccgtatggtaaaacggcagtcagaaaaataagtcataagattccgttatgaaaatatacttcggta
gttaataataagagatatgaggtaagagatacaagataagagatataaggtacgaatgtataagatggtgcttttaggcacactaa
ataaaaaacaaataaacgaaaattttaaggaggacgaaagacaagtttgtacaaaaaagctgaacgagaaacgtaaaatgatata
aatatcaatatattaaattagattttgcataaaaaacagactacataatactgtaaaacacaacatatccagtcactatg.

The sequence of pMetE_fix_A (pMU1728) is

(SEQ ID NO: 16)
ccgctcccggcggatttgtcctactcaggagagcgttcaccgacaaacaacagataaaacgaaaggcccagtctttc
gactgagccatcgttttatttgatgcctgggcgatcgtacttactgtttccccttctttaggcaatttgcttgatacaccaacttgtattct
tgttggatcatgtattaatattactttgcctttaaatctattacttgatatgtcgtatacttcaattgtgttatcatgagaatttgtaaaatttaa
tatatttttattgctactgcctgtagcgatattattagaatttttcatgatttcatctattttactctgaggcaagaataatgtaactatatattt
atgactaaaagttgtcattgcagatgtaactaatgtatttcttatatttgcgaatggcccataaaatatcaatacaggaattacaataatt
gataatatgaattcaaaaactaaatatacaataattcttttcgtcaaaatcatatttctcatagataactttcattcctttcatttataaacgg
catttatttttagtttaagttttttgggtgtcccatgttgtacatggtagttattcatagtatcctctgtaatatattagcataaaaaatattca
ggtatcaacaggaatttaaaaaattttcaaaaaatatattgactttataggtaaaccgcattatattaaataacatagtgttgcctattatt
tgctaaaagtattgtcatgtattgtaaaaaatctcattttagcttaatatatatttgtaattatatagtgtcggcttaaacatttgtttgatata
attattaataacaaaagttatattgattgggatggtagttatgattcagttaactgatacggaaattaaaaaaaggtgtgaaaatgata
gtgtctataaaagaggcattgaatattatttggcaggtaggatacacaattttacatacaacaaagctggcactgtatttcaagattt
gtgatgggcacatctttgtacagggtgatgatacaaaagtatcacggtgagttgtacacaagctgtacgagtcgtgactaagaacg
tcaaagtaattaacaatacagctatttttctcatgcttttacccctttcataaaatttaattttatcgttatcataaaaaattatagacgttata
ttgcttgccgggatatagtgctgggcattcgttggtgcaaaatgttcggagtaaggtggatattgatttgcatgttgatctattgcattg
aaatgattagttatccgtaaatattaattaatcatatcataaattaattatatcataattgttttgacgaatgaaggtttttggataaattatc
aagtaaaggaacgctaaaaattttggcgtaaaatatcaaaatgaccacttgaattaatatggtaaagtagatataatattttggtaaac
atgccttcagcaaggttagattagctgtttccgtataaattaaccgtatggtaaaacggcagtcagaaaaataagtcataagattccg
ttatgaaaatatacttcggtagttaataataagagatatgaggtaagagatacaagataagagatataaggtacgaatgtataagat
ggtgcttttaggcacactaaataaaaaacaaataaacgaaaattttaaggaggacgaaagatgatttcagttgtcggttttccaaga
ataggacaaaatagagagcttaaaaaatgggttgagagctatctggacaaaaatctttcaaaagaagagctcattcaaaactcaaa
aaacttaaaaaagactcactggcaacttcaaaaagagtatggtgttgacctgatatcatcaaatgacttttcgctttacgacactttttt
agaccatgcaatgcttgttggcgcaatacccgaggaatacaaggcggttttctcagatgatctcgagctctactttgcgcttgcaaa
gggatatcaagaccaaaacattgatcttaaagctttgcctatgaaaaagtggttctttacaaactaccactatcttgtgcctgaaatca
ctgaaaacaccaaatttgagctttcatcaacaaaaccttttgatgaatttgtcgaagcactttcaataggagttaagacaaaaccggc
aataatcggtgctctgacatttttaaagctttccaaaaaatcaaatgtggatatgtacgacaaatctttctgggaaaagctgcttgatgt
atatattcaaatactaaaaaggtttgaagagttaggtagcgagtttgttcagatagatgaaccgatacttgtcacagacttaagtaca
aaagacatagaattttttgaagatttttatcgcagtcttcttcttcataaaggaaagctgaaggtacttcttcagacctattttggagatg
tcagagactgcttcgaaaagataatctcccttgactttgacgcaattggccttgactttgttgatggaaagttcaatttagagctcatta
aaaaatttggttttccacaggataaptcctggttgctggagttgtaaatggcagaaatgtgtttaaaaacaactacaaaaatacgct
tgagcttttaaatatgctctcctcatttgttgacaagaaaaatattgtaatttcaacatcatgttccttactctttgtgccatactctttgaag
ttcgaaacacagcttgacagcaataaaaagaagtttttagcgtttgctgaggaaaagctaaaagagctgtctgagcttaagcttttgt
tctctcaagaaagctttaccgcaaacagcatctatgttcaaaatgttcagctttttgaagagctgaataaaaacaaactatcagatgtt
agcacagctgtaagtggtcttacagacgatgattttgaaagaaaaccctgttttgaagagagaatcaagcttcaaaaagaggttttg
aacttgccacagcttccgacaacaacaattgggtcattcccgcaaaccccggacgtgagggctgctcgaagcaagcttaaaaaa
ggtgaaataacacttgaagaatataaaaactttataaaatctaagattgaaagagtaataaagcttcaagaagaaatcgggcttgat
gtccttgtccacggcgaatacgaaagaaatgacatggtagagtttttcggtgaaaacttggaagggtttttaatcactcaaaacggt
tgggttcagtcatatggtacaagatgtgtaaaacctcctataatattttctgacattaaaagaaaaaaatcactcacagtggaatatat
aaaatacgcacaaagcttgacttcgaagcctgtaaaagggatcttgacaggaccagtgacaatcctcaactggtcatttgtgcgc
gaagatataccattgaaagatgtagcttttcagcttgctcttgcaataaaagaagaggttttggagcttgaaagagaaggtgtaaag
attattcagattgacgaggcagcactgattgaaaagcttccgctcaggcgctgccagcacagtagctatttgtcatgggcgataaa
agcattcaggctcacatgttcaaaagtaaaaccagaaactcaaattcatactcatatgtgttacagcaactttgatgagcttttagatg
aaatagcaaagatggatgtggacgttataacttttgaggcagctaaatctgattttacattgctcgacagcataaacaaaagtagttt
aaaagcagaggtaggtcctggcgtgtttgacgtgcattcacctcgaattgtatcaaaggaagagatgaaaaagctcatattaaaga
tgatagaaaaggttgggaaagacaggctgtgggtaaaccctgactgcggtcttaaaaccagaaaggaagaagaagttttgccta
ccttgcaaaacatggtgcttgcagcgtgggaagtcagaaataacttataatggagtttgtaatggatgtggccgactatttttacgtt
atggataaaggccgcatagtaatggagggaaaaacggagggaatcgatcctcatgaaatacaggaaaagattgctatttgataa
gtatgtcattgataaatatgccataaaattttgcgcctgtaaatttcgttgttaaaaatattacaaaaaaccaaaagcaatgaataagta
tttttagacagggaaaataaattttcctttggttatgccaatttatggattaatcaatttaaaagaaggtggtaagagtgcatttgacgc
ccagggaaaccgaaaaattgatgcttcattatgccggtgaactggcaagaaaacgaaaagaaagaggtcttaagcttaattatcc
ggaagctgtagcccttataagcgctgaactgatggaggccgcccgggacggaaaaactgtaacggaactgatgcagtatggag
caaagatactgaccagggatgatgtaatggaaggagttgacgccatgatacatgaaattcagatagaggcaactttcccggacg
gtacaaagcttgttaccgttcacaatcctatacgctagagggaggaaggatgtatgattcctggcgagtacattataaaaaatgagt
ttatcacattgaatgatggaagaaggactttaaatatcaaggtttcaaatacaggagaccggcccgttcaggtggggtcccactac
catttcttcgaagttaatcggtatcttgagtttgacagaaaaagcgctttcggaatgagactggacattccttcgggtactgcggtaa
ggtttgagccgggggaggaaaagacagttcaactggttgaaatagggggaagcagagaaatttacggacttaatgatctgactt
gcggtccccttgacagagaagatttgtccaatgtgtttaaaaaggcgaaagagctggggttcaagggggtggaataacatgagt
gtaaaaataagcggcaaagattatgccggtatgtatggcccgacaaaaggcgacagggtgaggctggcagacacggatctcat
tattgagattgaggaagattacacggtttatggagatgagtgcaaattcggaggaggtaaatccataagggacggaatgggcca
gtctccttcggctgcaagagatgacaaggttttggatttggtaattaccaatgccataatctttgacacatgggggattgtaaaggga
gatataggtataaaagacggaaaaatagccggaatcgggaaggcgggaaatccgaaagtaatgagcggcgtgtcggaggatt
taataatcggggcctctaccgaagttattaccggagaaggacttattgtgactccgggaggaattgatacacatatacattttatatg
cccccagcagattgagaccgcattgttcagcggtatcacaacaatgattggtggcggaacgggaccggcagacggaaccaatg
ccaccacttgcacaccgggagcctttaacatccggaaaatgttagaggcggcagaggactttccggtaaatttaggttttttgggg
aaagggaatgcttcttttgagactcctctgatagaacagattgaagcaggggcgattggcttaaagctccatgaggattggggaa
ccacacccaaggctatagatacatgcctgaaagttgcggatctttttgatgtacaggtggctatacataccgatacactgaacgag
gcaggatttgtagagaatactatagcggctatagccggaaggacaattcacacttaccataccgagggagcgggcggcgggca
cgcaccggacataattaaaattgcatcacgcatgaatgtactgccctcgtctaccaatcccaccatgccttttaccgtcaatacattg
gatgaacatctcgatatgcttatggtatgccatcatcttgacagcaaggtaaaagaggacgttgcttttgccgattcgaggatccgg
cctgagacaatagccgcagaagacatactgcacgatatgggagtattcagcatgatgagttccgattcccaggccatgggacgc
gtgggagaggttattataaggacctggcagactgcacataaaatgaagcttcaaagaggtgccctgccgggggaaaagagcg
gctgtgacaatataagggctaaaagataccttgccaagtataccataaaccctgctataacccatggaatttcacagtatgtgggct
ccctggagaaagggaaaatagccgacttggtcctctggaagcctgcaatgtttggtgtaaagcctgaaatgattattaagggcgg
ctttataatagccggcaggatgggcgatgcaaatgcgtccatacccacacctcagcctgtaatatataaaaacatgttcggtgcctt
cggaaaggcaaagtacggaacctgtgtgacttttgtttcaaaggcttcgctggaaaatggcgttgtggaaaagatggggcttcaa
agaaaagtgcttccggtccagggatgcaggaatatctcaaaaaaatatatggtacacaacaatgcaacgcctgaaattgaagttg
atcctgaaacctatgaggtaaaggtggacggtgagattatcacctgcgaaccattaaaggtcttacccatggcgcagagatatttc
ttgttttaaactgccggaaggttagtttctctgtaaaaaatttatggtaattgacatttcaaaaaacaattttaaactaaagaaatttttaa
ataaagaataattttgggaggacttaaaaaaaactcaaaaacataagttgggtgagatgaaatgattgttgaaagagttttgtataat
atcaaagatatcgacttggaaaaattggaagttgatttcgtggatattgaatggtatgaagttcaannaaaaatactacgcssattaa
gttccaacggaattgaagttggaataagaaacagcaacggtgaggctttaaaagaaggagacgtattgtggcaggagggaaat
aaagttttggttgtaaggattccctattgcgactgtatcgtgctgaagcctcaaaatatgtatgagatgggcaagacttgctatgaga
tgggaaacagacatgcacctctttttattgatggagatgagctgatgactccctatgatgagccgttgatgcaggcattgataaaat
gcgggctttcaccttacaaaaagagctgtaaacttacaacgcccttaggaggtaatcttcatggatactcccattctcattcccactg
atatgaatagaataccctttttttaccttttacagattagcgatccgctgtttccgataggaggttttacccaatcctatgggcttgaaac
ctatgtgcaaaaagggattgtccatgatgctgaaacttcgaaaaaataccttgaaagctatcttttaaacagctttttgtacaatgattt
attggccgtcaggctttcctgggaatatacccaaaaaggaaatttgaataaggtattggaactttcggaagttttttcggcctcaaag
gcgccgagggagcttagagcggcaaatgaaaagctcggcaggaggtttataaagatactggaatttgttttgggcgaaaacgaa
atgttttgcgaaatgtatgaaaaagtggggagaggaagtgtggaagtttcgtatcctgtaatgtacggtttttgtacaaatcttctcaa
tatcggaaaaaaggaagcgttgtcggcggttacttatagcgcggcatcttccataataaataactgtgcaaaattggtacctatcag
ccagaacgaagggcagaagattttattcaatgcccatggcattttccgaaggcttttggaaagagtggaggaactggacgagga
atatctgggaagctgctgctttggatttgacttaagagccatgcagcatgaaaggctctatacaaggctttatatatcctagtgttaat
aatcctgtactacattgttatttatcttcttaaggaaggtggagcttatgaattatgtgaaaatcggcgtgggaggtccggtaggatcg
ggcaagaccgcccttatagaaaaattgacaagaatattggctgattcttacagcatcggggtggttaccaacgatatatacacaaa
agaggacgcggaatttttaataaagaacagtgtacttcccaaagagaggataattggagtggaaaccggcggctgccctcatac
ggctattcgcgaggatgcttccatgaaccttgaagctgtggaggaactggtacagcggttccctgatattcaaattgtgtttattgaa
agcgggggagacaatctttccgcaactttcagtccggaactggccgatgccaccatatatgtcatcgatgtggccgaaggtgaca
aaattccccgaaaaggcggcccgggaataacccggtcggatttactggtcataaataaaattgatctggctccatacgtgggagc
aagccttgaggtaatggaaagggattcaaagaagatgaggggtgagaaaccttttatattcaccaatttgaatacaaatgaaggtg
tggataagattatcgattggattaagaaaagcgtccttttggaaggtgtgtaaattatgaagaataaattcggaaaagaaagcaggc
tgtacataagagcaaaggtttcagacggaaaaacatgccttcaggattcgtatttcacagcaccttttaaaatagccaaaccctttta
tgaagggcatggcggatttatgaatcttatggttatgtcagcttcagcgggagttatggagggtgacaattacaggattgaagtgg
aattggacaaaggcgcaagagtgaaactggaaggccagtcctaccagaagattcaccggatgaaaaatggaacggcagtgca
gtacaacagttttacccttgcagacggagcgtttttggattatgctcccaaccccaccataccttttgccgactcagcattttattcaaa
tacagaatgcaggatggaagaaggctcagcctttatctattcggagatactggccgcgggcagggttaagagcggtgaaattttc
cggttcagggaatatcacagcgggataaagatttattacggcggggaactgatttttcttgaaaatcagttcctttttccaaaagtgc
agaatcttgaaggaatcggattttttgaaggttttacacatcaggcgtcaatgggttttttttgtaagcagataagcgatgaacttattg
ataaactttgtgtaatgcttacggccatggaggatgtccagttcggattgagcaaaacaaagaagtatggctttgttgttcggattct
cggaaacagcagtgataggctggaaagtattctaaaactgattagaaatatcctctattagtaaaaataaacactatttttggttatga
aaatcagaactaaatgtttttggcagtataaaactgtaaaaacggtttaaaaaaagaaagtgtacaagcattgaaaaatatcaactgtt
aaaaaagttgtaatttagagatgagccggttgttgaaaagttgaatgcccaaatcccgttaagttatatcttaatcggaaaaaagaat
aaaagaaattcgatttatgataaaataccttgacaattttggattacagctgtaagatataattagacttacaattgtaatctaaaatgg
aggggcaattatgaaagcagagtctcaaatcacagaagcggaactggaagttatgaaaattctttgggagtatggaaaggccac
cagttctcagatcgtgcccattgtgaagtggattgtattctacaattaaacctaatacgctcataatatgcgcctttctaaaaaattatta
attgtacttattattttataaaaaatatgttaaaatgtaaaatgtgtatacaatatatttcttcttagtaagaggaatgtataaaaataaatat
tttaaaggaagggacgatcttatgagcattattcaaaacatcattgaaaaagctaaaagcgataaaaagaaaattgttctgccagaa
ggtgcagaacccaggacattaaaagctgctgaaatagttttaaaagaagggattgcagatttagtgcttcttggaaatgaagatga
gataagaaatgctgcaaaagacttggacatatccaaagctgaaatcattgaccctgtaaagtctgaaatgtttgataggtatgctaat
gatttctatgagttaaggaagaacaaaggaatcacgttggaaaaagccagagaaacaatcaaggataatatctattttggatgtatg
atggttaaagaaggttatgctgatggattggtatctggcgctattcatgctactgcagatttattaagacctgcatttcagataattaaa
acggctccaggagcaaagatagtatcaagcttttttataatggaagtgcctaattgtgaatatggtgaaaatggtgtattcttgtttgct
gattgtgcggtcaacccatcgcctaatgcagaagaacttgcttctattgccgtacaatctgctaatactgcaaagaatttgttgggctt
tgaaccaaaagttgccatgctatcattttctacaaaaggtagtgcatcacatgaattagtagataaagtaagaaaagcgacagagat
agcaaaagaattgatgccagatgttgctatcgacggtgaattgcaattggatgctgctcttgttsaagaagttgcagagctaaaagc
gccgggaagcaaagttgcgggatgtgcaaatgtgcttatattccctgatttacaagctggtaatataggatataagcttgtacagag
gttagctaaggcaaatgcaattggacctataacacaaggaatgggtgcaccggttaatgatttatcaagaggatgcagctataga
gatattgttgacgtaatagcaacaacagctgtgcaggctcaataaaatgtaaagtatggaggatgaaaattatgaaaatactggtta
ttaattgcggaagttcttcgctaaaatatcaactgattgaatcaactgatggaaatgtgttggcaaaaggccttgctgaaagaatcgg
cataaatgattccatgttgacacataatgctaacggagaaaaaatcaagataaaaaaagacatgaaagatcacaaagacgcaata
aaattggttttagatgctttggtaaacagtgactacggcgttataaaagatatgtctgagatagatgctgtaggacatagagttgttca
cggaggagaatcttttacatcatcagttctcataaatgatgaagtgttaaaagcgataacagattgcatagaattagctccactgcac
aatcctgctaatatagaaggaattaaagcttgccagcaaatcatgccaaacgttccaatggtggcggtatttgatacagcctttcatc
agacaatgcctgattatgcatatctttatccaataccttatgaatactacacaaagtacaggattagaagatatggatttcatggcaca
tcgcataaatatgtttcaaatagggctgcagagattttgaataaacctattgaagatttgaaaatcataacttgtcatcttggaaatggc
tccagcattgctgctgtcaaatatggtaaatcaattgacacaagcatgggatttacaccattagaaggtttggctatgggtacacgat
ctggaagcatagacccatccatcatttcgtatcttatggaaaaagaaaatataagcgctgaagaagtagtaaatatattaaataaaa
aatctggtgtttacggtatttcaggaataagcagcgattttagagacttagaagatgccgcctttaaaaatggagatgaaagagctc
agttggctttaaatgtgtttgcatatcgagtaaagaagacgattggcgcttatgcagcagctatgggaggcgtcgatgtcattgtatt
tacagcaggtgttggtgaaaatggtcctgagatacgagaatttatacttgatggattagagtttttagggttcagcttggataaagaa
aaaaataaagtcagaggaaaagaaactattatatctacgccgaattcaaaagttagcgtgatggttgtgcctactaatgaagaatac
atgattgctaaagatactgaaaagattgtaaagagtataaaatagcattatgacaaatgtttaccccattagtataattaattttggca
attatattggggtgagaaaatgaaaattgatttatcaaaaattaaaggacataggggccgcagcatcgaagtcaactacgtaaaac
ccagcgaaccatttgaggtgataggtaagattataccgaggtatgaaaacgagaattggacctttacagaattactctatgaagcg
ccatatttaaaaagctaccaagacgaagaggatgaagaggatgaggaggcagattgccttgaatatattgacaatactgataaga
taatatatcttttatatagaagatatcgccgtatgtaaggatttcagggggcaaggcataggcagcgcgcttatcaatatatctatag
aatgggcaaagcataaaaacttgcatggactaatgcttgaaacccaggacaataaccttatagcttgtaaattctatcataattgtgg
tttcaaaatcggctccgtcgatactatgttatacgccaactttcaaaacaactttgaaaaagctgttttctggtatttaaggttttagaat
gcaaggaacagtgaattggagttcgtcttgttataattagcttcttggggtatctttaaatactgtagaaaagaggaaggaaataata
aatggctaaaatgagaatatcaccggaattgaaaaaactgatcgaaaaataccgctgcgtaaaagatacggaaggaatgtctcct
gctaaggtatataagctggtgggagaaaatgaaaacctatatttaaaaatgacggacagccggtataaagggaccacctatgatg
tggaacgggaaaaggacatgatgctatggctggaaggaaagctgcctgttccaaaggtcctgcactttgaacggcatgatggct
ggagcaatctgctcatgagtgaggccgatggcgtcctttgctcggaagagtatgaagatgaacaaagccctgaaaagattatcg
agctgtatgcggagtgcatcaggctctttcactccatcgacatatcggattgtccctatacgaatagcttagacagccgcttagccg
aattggattacttactgaataacgatctggccgatgtggattgcgaaaactgggaagaagacactccatttaaagatccgcgcgag
ctgtatgattttttaaagacggaaaagcccgaagaggaacttgtcttttcccacggcgacctgggagacagcaacatctttgtgaa
agatggcaaagtaagtggctttattgatcttgggagaagcggcagggcggacaagtggtatgacattgccttctgcgtccggtcg
atcagggaggatatcggggaagaacagtatgtcgagctattttttgacttactggggatcaagcctgattgggagaaaataaaata
ttatattttactggatgaattgttttagtacctagatttagatgtctaaaaagctttttagacatctaatcttttctgaagtacatccgcaact
gtccatactctgatgttttatatcttttctaaaagttcgctagataggggtcccgagcgcctacgaggaatttgtatcggaagatcaag
cgacagatagagcccacaggattgggcaggttaatacagtacaagtcataaagcttataacgcaaggtacaattgaagaaaaaa
ttgtaaagctgcaagagaagaaaaaagagatgataaattctgtcataaatccaggtgaaacgtttataactaagttgagtgaagaa
gaagtaaaagagctttttgcaatgtgatttaatgatttgcaattgccgattaaggcagttgctttttttatgttacaagattgtaatagaaa
attaaggaataattaataaaatttataattttaaattttataatagagatgaggcatgggaggttaagagtataatctatattgataaaag
tcactttgtctgggaggctattatgaataaagtgaaactatgtttattaattatcgtaatcttaatacttggtggctgtagtattaaaagta
caaatacagacttaagcaatgataatataattattgataaaacaaatggtaatatacttgatgagttagaggataaaaagacctcatc
gattgaaaatgcacatccaatagctgtgcttgatgatggcagaaaagtgtttttgcaggtcaatcctgaagttgacaacagcattttt
gttacctcaagtgacagctcaataatttttaaaattaatgctggaatttctaaaaatatttatgatgcaaaagtcatggggaattggatc
gtgtatgttgaatccagcaacgatatgacaaaaagcgattgggctttgtatgctaaaaatatagatgacaatcgtcgcatagaaatt
gataaaggaaatgttgtaaatgcaaaagtaaaaacgcctactttgttaggagcgttgatagctgcatctctatcagctgtccctcctg
ttcagctactgacggggtggtgcgtaacggcaaaagcaccgccggacatcagcgctagcggagtgtatactggcttactatgttg
gcactgatgagggtgtcagtgaagtgcttcatgtggcaggagaaaaaaggctgcaccggtgcgtcagcagaatatgtgatacag
gatatattccgcttcctcgctcactgactcgctacgctcggtcgttcgactgcggcgagcggaaatggcttacgaacggggcgga
gatttcctggaagatgccaggaagatacttaacagggaagtgagagggccgcggcaaagccgtttttccataggctccgccccc
ctgacaagcatcacgaaatctgacgctcaaatcagtggtggcgaaacccgacaggactataaagataccaggcgtttccccctg
gcggctccctcgtgcgctctcctgttcctgcctttcggtttaccggtgtcattccgctgttatggccgcgtttgtctcattccacgcctg
acactcagttccgggtaggcagttcgctccaagctggactgtatgcacgaaccccccgttcagtccgaccgctgcgccttatccg
gtaactatcgtcttgagtccaacccggaaagacatgcaaaagcaccactggcagcagccactggtaattgatttagaggagttag
tcttgaagtcatgcgccggttaaggctaaactgaaaggacaagttttggtgactgcgctcctccaagccagttacctcggttcaaa
gagttggtagctcagagaaccttcgaaaaaccgccctgcaaggcggttttttcgttttcagagcaagagattacgcgcagaccaa
aacgatctcaagaagatcatcttattaatcagataaaatatttctagatttcagtgcaatttatctcttcaaatgtagcacctgaagtcag
ccccatacgatataagttgtaattctcatgtttgacagcttatcatcgataagctttaatgcggtagtttatcacagttaaattgctaacg
cagtcaggcacctatacatgcatttacttataatacagttttttagttttgctggccgcatcttctcaaatatgcttcccagcctgcttttct
gtaacgttcaccctctaccttagcatcccttccctttgcaaatagtcctcttccaacaataataatgtcagatcctgtagagaccacat
catccacggttctatactgttgacccaatgcgtctcccttgtcatctaaacccacaccgggtgtcataatcaaccaatcgtaaccttc
atctcttccacccatgtctctttgagcaataaagccgataacaaaatctttgtcgctcttcgcaatgtcaacagtacccttagtatattct
ccagtagatagggagcccttgcatgacaattctgctaacatcaaaaggcctctaggttcctttgttacttcttctgccgcctgcttcaa
accgctaacaatacctgggcccaccacaccgtgtgcattcgtaatgtctgcccattctgctattctgtatacacccgcagagtactg
caatttgactgtattaccaatgtcagcaaattttctgtcttcgaagagtaaaaaattgtacttggcggataatgcctttagcggcttaac
tgtgccctccatggaaaaatcagtcaagatatccacatgtgtttttagtaaacaaattttgggacctaatgcttcaactaactccagta
attccttggtggtacgaacatccaatgaagcacacaagtttgtttgcttttcgtgcatgatattaaatagcttggcagcaacaggacta
ggatgagtagcagcacgttccttatatgtagctttcgacatgatttatcttcgtttcctgcaggatttgttctgtgcagttgggttaaga
atactgggcaatttcatgtttcttcaacactacatatgcgtatatataccaatctaagtctgtgctccttccttcgttcttccttctgttcgg
agattaccgaatcaaaaaaatttcaaagaaaccgaaatcaaaaaaaagaataaaaaaaaaatgatgaattgaattgaaaagctag
cttatcgatgggtccttttcatcacgtgctataaaaataattataatttaaattattaatataaatatataaattaaaaatagaaagtaaaa
aaagaaattaaagaaaaaatagtttttgttttccgaagatgtaaaagactctagggggatcgccaacaaatactaccttttatcttgct
cttcctgctctcaggtattaatgccgaattgtttcatcttgtctgtgtagaagaccacacacgaaaatcctgtgattttacattttacttat
cgttaatcgaatgtatatctatttaatctgcttttcttgtctaataaatatatatgtaaagtacgctttttgttgaaattattaaacctttgttta
tttttttttcttcattccgtaactcttctaccttctttatttactttctaaaatccaaatacaaaacataaaaataaataaacacagagtaaatt
cccaaattattccatcattaaaagatacgaggcgcgtgtaagttacaggcaagcgatctctaagaaaccattattatcatgacattaa
cctataaaaaaggcctctcgagctagagtcgatcttcgccagcagggcgaggatcgtggcatcaccgaaccgcgccgtgcgcg
ggtcgtcggtgagccagagtttcagcaggccgcccaggcggcccaggtcgccattgatgcgggccagctcgcggacgtgctc
atagtccacgacgcccgtgattttgtagccctggccgacggccagcaggtaggccgacaggctcatgccggccgccgccgcc
ttttcctcaatcgctcttcgttcgtctggaaggcagtacaccttgataggtgggctgcccttcctggttggcttggtttcatcagccatc
cgcttgccctcatctgttacgccggcggtagccggccagcctcgcagagcaggattcccgttgagcaccgccaggtgcgaata
agggacagtgaagaaggaacacccgctcgcgggtgggcctacttcacctatcctgcccggctgacgccgttggatacaccaag
gaaagtctacacgaaccctttggcaaaatcctgtatatcgtgcgaaaaaggatggatataccgaaaaaatcgctataatgacccc
gaagcagggttatgcagcggaaaagcgctgcttccctgctgattgtggaatatctaccgactggaaacaggcaaatgcaggaa
attactgaactgaggggacaggcgagagacgatgccaaagagctacaccgacgagctggccgagtgggttgaatcccgcgc
ggccaagaagcgccggcgtgatgaggctgcggttgcgttcctggcggtgagggcggatgtcgatatgcgtaaggagaaaata
ccgcatcaggcgcatatttgaatgtatttagaaaaataaacaaaaagagtttgtagaaacgcaaaaaggccatccgtcaggatgg
ccttctgcttaatttgatgcctggcagtttatggcgggcgtcctgcccgccaccctccgggccgttgcttcgcaacgttcaaat.

Using genetic methods previously established, including transformation, positive selection, and marker removal, the above plasmids were used to create two urease⁺ strains of T. saccharolyticum. T. saccharolyticum JW/SL-YS485, strain M0863 carrying deletion of L-lactate dehydrogenase (L-ldh), phosphoacetyltransferase (pta), and acetate kinase (ack) was used as the host strain for this work. T. saccharolyticum transformed with pDest-Ct-urease (pMU1336) (SEQ ID NO: 15) is referred to as strain M1051. Plasmid pMU1366 is a non-replicating plasmid which integrates into the chromosome a the ΔL-ldh locus. The Gateway® cloning system (Invitrogen) was used according to the manufacturer's instructions in the creation of the M1051 strain. T saccharolyticum transformed with pMetE_fix_A (pMU1728) (SEQ ID NO: 16) is referred to as strain M1151. Plasmid pMU1728 is a non-replicating plasmid which integrates into the chromosome at the orf796 locas. Strains M1051 (ATCC deposit designation PTA-10494) and M1151 (ATCC deposit designation PTA-10495) were deposited at the ATCC on Nov. 24, 2009.

For the following Examples in which the M1051 (urease⁺) strain was compared to the M0863 (urease⁻) strain, TSD1 media formulations (as shown in Table 2) were used. 1.85 g/L ammonium sulfate was replaced with 2 g/L urea to make urea containing media as required in each experiment.

TABLE 2
TSD1 Base Medium

| | | Concen- | | |
|---|---|---|---|---|
| | | tration, | | Batch |
| Solutions | Components | g/l | Manufacturer | Number |
| Solution I | (NH₄)₂SO₄ | 1.85 | Sigma A4418 | 068K54412 |
| (Mineral | FeSO₄*7H2O | 0.05 | Sigma F8633 | 023K06151 |
| Solution) | KH₂PO₄ | 1.0 | Sigma P5655 | 097K0067 |
| | MgSO₄ | 1.0 | Sigma | 036K00251 |
| | | | M2643 | |
| | CaCl₂*2H₂O | 0.1 | Sigma 223506 | 10729LD |
| | Trisodium citrate | 2 | Sigma C8532 | 087K0055 |
| | * 2 H₂O | | | |
| Solution | p-Amino | 0.002 | Sigma A9878 | 036K1339 |
| II | Benzoic Acid | | | |
| (Flamingo | Thiamine•HCl | 0.002 | Sigma T1270 | 095K07031 |
| Red | Vitamin B12 | 0.00001 | Sigma V2876 | 106K1087 |
| Solution) | L-Methionine | 0.12 | Fisher BP388 | 045593 |

For the following Examples, in which the M1151 (urease⁺) strain was compared to the M0863 (urease⁻) strain, TSC2 media formulations (as shown in Table 3), were used. 8.5 or 0.5 g/L yeast extract was added as required in each experiment.

TABLE 3
TSC2 Base Medium

| | | |
|---|---|---|
| | Final | |
| Components | Concentration, g/l | Manufacturer |

Solution I

| | | |
|---|---|---|
| Maltodextrin | 75 | Fluka 31410 |
| Cellobiose | 75 | Sigma C7252 |
| CaCO₃ | 7.5 | Sigma 310034 |

Solution II

| | | |
|---|---|---|
| (NH₄)₂SO₄ | 1.85 | Sigma A4418 |
| FeSO₄*7H2O | 0.1 | Sigma F8633 |
| KH₂PO₄ | 2.0 | Sigma P5655 |
| MgSO₄ | 2.0 | Sigma M2643 |
| CaCl₂*2H₂O | 0.2 | Sigma 223506 |
| Trisodium citrate | 4 | Sigma C8532 |
| * 2 H₂O | | |
| Yeast Extract | 8.5 | BD Difco Low Dust 210941 |
| Methionine | 0.12 | Sigma A9878 |
| L-Cysteine HCl | 0.5 | Sigma C7880 |

### Example 2

### Pressure Recordings of Fermentations

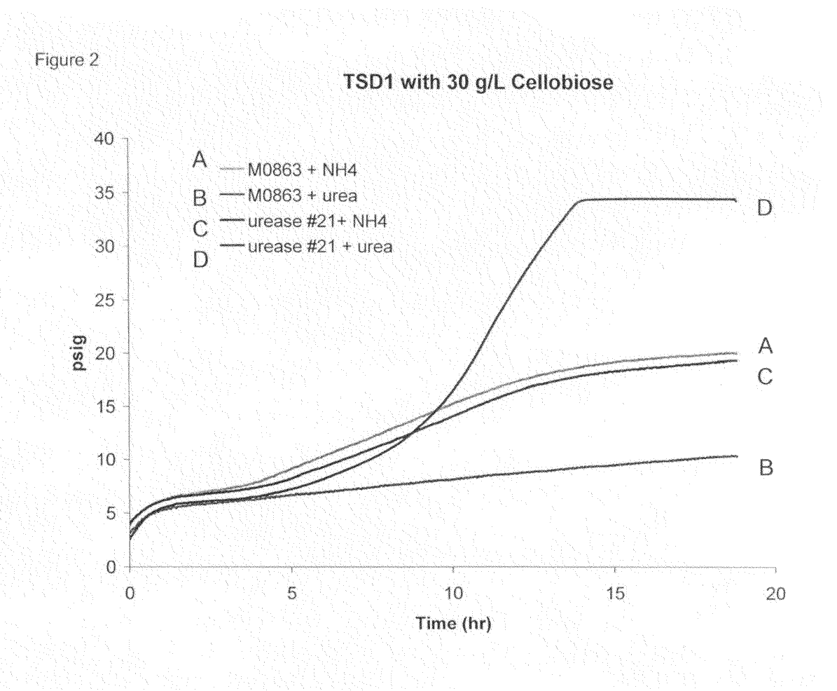

In order to determine the ability of the transformed T. saccharolyticum to use urea as a nitrogen source, pressure recording of fermentations were performed with strains M0863 (L-ldh− pta/ack−) and M1051 (L-ldh− pta/ack− urease+) in TSD1 medium containing 30 g/L of cellobiose and additionally with either ammonium sulfate or urea as nitrogen source. Pressure recordings were performed in sealed serum bottles punctured by a hypodermic luer-lock needle attached to a pressure transducer. The results are shown in FIG. 2.

Neither M1051 nor M0863 cells using ammonium as a nitrogen source exceeded 20 psig over the time of the experiment (20 hours). M0863 cells using urea as a nitrogen source never exceeded 10 psig over the same period. However, M1051 cells using urea as a nitrogen source peaked at over 35 psig during the period of measurement.

### Example 3

### Fermentation Performance

In order to determine the ability of the transformed T. saccharolyticum to use urea as a nitrogen source, fermentation performance was evaluated through measurement of various indicators of fermentation.

Table 4 (below) depicts measurements of the fermentation indicator ethanol (EtOH), as well as OD (optical density) and pH after 19 hours of growth. Strains M0863 (L-ldh− pta/ack−) and M1051 (L-ldh− pta/ack− urease+) were tested in TSD1 medium containing 30 g/L of cellobiose and additionally with either ammonium sulfate or urea as nitrogen source. M0863 cells using ammonium as a nitrogen source produced 5.2 g/L of EtOH. M1051 cells using ammonium as a nitrogen source produced 4.7 g/L of EtOH. M0863 cells tested with urea as a nitrogen source only produced 2.0 g/L of EtOH, whereas M1051 cells, in contrast, produced 11.5 g/L of EtOH. The final pH of ammonium contains M0863 and M1051 fermentations was 3.58 and 3.48, respectively, while the final pH of urea containing fermentations was 4.37 and 5.45 for M0863 and M1051.

TABLE 4

| | | | |
|---|---|---|---|
| M0863 + | M0863 + | M1051 + | M1051 + |
| NH4 | urea | NH4 | urea |

Initial time - 0 hours

| | | | | |
|---|---|---|---|---|
| CB (g/L) | 28.1 | 27.9 | 28.0 | 27.8 |
| G (g/L) | 0.2 | 0.3 | 0.2 | 0.3 |

Final time - 19 hours

| | | | | |
|---|---|---|---|---|
| CB (g/L) | 15.9 | 23.2 | 16.8 | 0.4 |
| G (g/L) | 0.0 | 0.1 | 0.0 | 0.0 |
| Etoh (g/L) | 5.2 | 2.0 | 4.7 | 11.5 |
| OD | 3.9 | 0.9 | 4.3 | 6.4 |
| pH | 3.58 | 4.37 | 3.48 | 5.45 |
| Etoh yield | 0.43 | 0.43 | 0.42 | 0.42 |
| g/g | | | | |
| Cell yield | 0.16 | 0.10 | 0.19 | 0.12 |
| g/g | | | | |

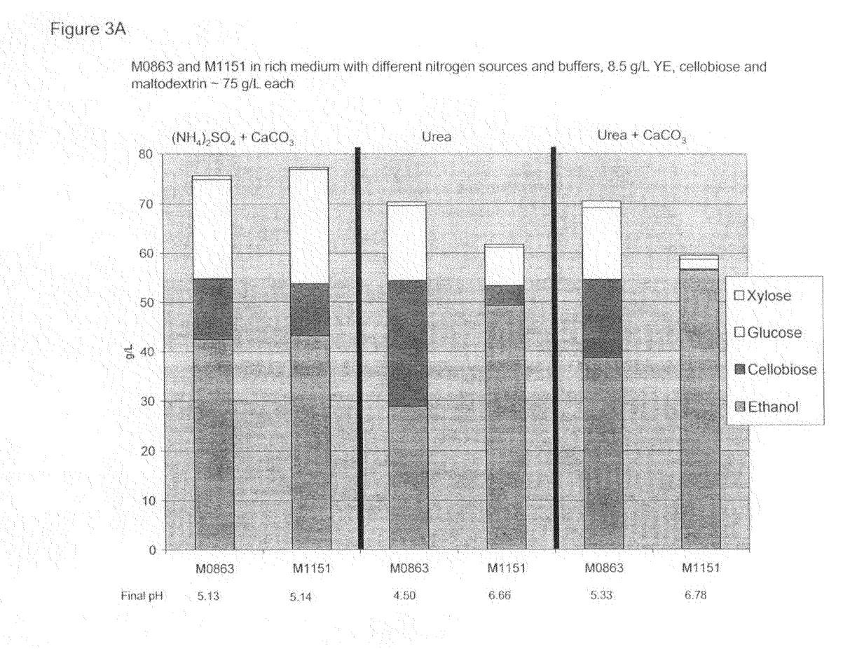

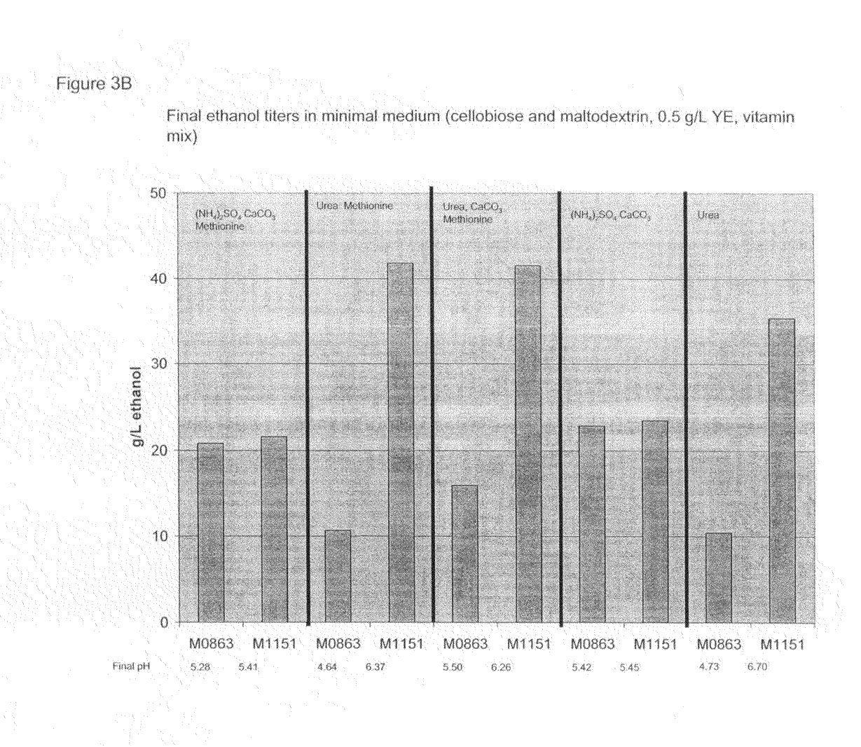

FIG. 3A depicts the fermentation performance of strains M0863 (L-ldh− pta/ack) and M1151 (L-ldh− pta/ack−, urease+, metE+, or 796−) in high yeast extract (i.e. 8.5 g/L) rich medium, cellobiose (about 75 g/L), and maltodextrin (about 75 g/L). The strains were grown with different nitrogen sources and presence or absence of CaCO₃ buffering. Fermentation performance was measured by the amount of ethanol (EtOH), Cellobiose (CB), Glucose, and Xylose present after 96 hours of fermentation. All cultures were grown at 55° C. with shaking at 150 rpm. Fermentations were performed in 150 mL serum bottles with a 20 mL culture volume, and bottles were sealed with butyric rubber stoppers after evacuation of air and replacement with an atmosphere containing 95% nitrogen and 5% carbon dioxide.

M0863 converted the most cellobiose into EtOH when ammonium sulfate and CaCO₃ were added to the growth media. M0863 cells converted the least amount of cellobiose into EtOH when urea was added to the growth media. The M1151 strain converted cellobiose and maltodextrin into EtOH at a final titer of 56 g/L when urea and CaCO₃ buffer were added to the growth media. Without the CaCO₃ buffer, M1151 cells were slightly less efficient at converting cellobiose into EtOH. Using ammonium sulfate as a nitrogen source, the M1151 strain's efficiency at cellobiose fermentation into EtOH was equivalent to that of the M0863 strain, at 43-45 g/L EtOH.

FIG. 3B depicts ethanol (EtOH) production by M0863 and M1151 grown in low yeast extract (i.e. 0.5 g/L) rich medium with cellobiose (about 75 g/L), maltodextrin (about 75 g/L), and vitamins. The strains were grown with different nitrogen sources and presence or absence of CaCO₃ buffering, as discussed below. M0863 cells produced the most EtOH when grown in the above-described media with ammonium sulfate as a nitrogen source and the presence of CaCO₃ buffer. M0863 cells produced the least EtOH when grown in media supplemented with urea only. The addition of methionine had very little effect on the production of EtOH by M0863 cells grown under either condition. M1151 cells produced the most EtOH when grown in media with urea and methionine. EtOH production by these cells was slightly less when urea, methionine and a buffer were included in the growth media. The addition of urea allowed for the production of over 30 g/L of EtOH by M1151 cells. When the ammonium sulfate was used as a nitrogen source, the production of EtOH was equivalent between the M0863 and M1151 strains.

### Example 4

### Expression of Urease Genes in a T. saccharolyticum Strain Producing Organic Acids

Plasmid pMU1728 was transformed into wildtype T. saccharolyticum cells, creating a stain carrying the urease operon, the MetE gene, and two copies of the pta and ack genes (the wildtype copy and a recombinant copy). In addition to acetic acid, this strain, M1447, is also able to produce lactic acid and ethanol. Utilization of urea allows for a higher pH during ethanol and organic acid production, as well as a final higher product titer in the urea utilizing strain. Batch fermentations were run in 15 mL falcon tubes with a 5 mL working volume for 7 days at 55° C. without shaking in an anaerobic chamber. Analysis was performed at the fermentation endpoint, and on un-inoculated media. The results are shown in Table 5 below and demonstrate that the highest levels of lactic acid, acetic acid, and ethanol were produced by M1447 in the presence of urea.

TABLE 5

| | | | | | | | | Carbon |
|---|---|---|---|---|---|---|---|---|
| | | | | | | | | Recov- |
| | CB | G | X | LA | AA | Etoh | pH | ery % |
| TSC4 | 29.99 | 0.19 | 4.91 | 0.00 | 0.00 | 0.21 | 5.80 | 100 |
| media | | | | | | | | |
| M0010 | 21.09 | 1.70 | 2.17 | 1.62 | 2.32 | 3.14 | 4.42 | 101 |
| (wt) | | | | | | | | |
| M1447 | 0.38 | 0.48 | 0.82 | 2.62 | 4.55 | 12.75 | 7.89 | 97 |
| (wt + | | | | | | | | |
| pMU1728) | | | | | | | | |
| TSD1 | 13.11 | 0.00 | 4.04 | 0.00 | 0.00 | 0.00 | 6.10 | 100 |
| media | | | | | | | | |
| M0010 | 6.29 | 4.39 | 2.70 | 0.90 | 0.71 | 1.26 | 4.73 | 102 |
| (wt) | | | | | | | | |
| M1447 | 0.00 | 0.00 | 0.00 | 1.91 | 1.24 | 6.62 | 6.74 | 94 |
| (wt + | | | | | | | | |
| pMU1728) | | | | | | | | |

The TSC4 media used in these experiments was prepared as described in Table 6.

TABLE 6
TSC4 Medium

| | |
|---|---|
| Components | Final Concentration, g/l |

Solution I

| | |
|---|---|
| D-(+) Xylose | 5 |
| Cellobiose | 30 |

Solution II

| | |
|---|---|
| Yeast Extract | 8.5 |
| Trisodium citrate * 2 H₂O | 4 |
| KH₂PO₄ | 2 |
| MgSO₄*7H₂O | 2 |
| Urea | 5 |
| CaCl₂*2H₂O | 0.2 |
| FeSO₄*7H₂O | 0.2 |
| Methionine | 0.12 |
| L-Cysteine HCl | 0.5 |

Solution 1 is prepared at 1.1× final concentration and autoclaved, while solution 2 is prepared at 10× concentration and filter sterilized. Solutions 1 and 2 are then combined under an anaerobic atmosphere.

These examples illustrate possible embodiments of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A recombinant anaerobic, thermophilic host cell comprising one or more heterologous polynucleotides encoding (a) at least two catalytic subunits of a urease enzyme and (b) four urease accessory proteins.

2. The recombinant anaerobic, thermophilic host cell of claim 1, wherein said host is of the genus Thermoanaerobacter or Thermoananerbacterium.

3. The recombinant anaerobic, thermophilic host cell of claim 2, wherein said host is T. saccharolyticum.

4. The recombinant anaerobic, thermophilic host cell of claim 1, wherein said host heterologously expresses three catalytic subunits of a urease enzyme.

5. The recombinant anaerobic, thermophilic host cell of claim 1, wherein said catalytic subunits are selected from group consisting of urease α, β and γ.

6. The recombinant anaerobic, thermophilic host cell of claim 1, wherein said accessory proteins are urease D, E, F, and G.

7. The recombinant anaerobic, thermophilic host cell of claim 1, wherein said urease catalytic subunits and accessory proteins are derived from an anaerobic, thermophilic organism that natively expresses the urease enzyme.

8. The recombinant anaerobic, thermophilic host cell of claim 1, wherein said urease catalytic subunits and accessory proteins are derived from Clostridium thermocellum.

9. The recombinant anaerobic, thermophilic host cell of claim 1, wherein nickel in the host cell is captured by the metallochaperone ureE.

10. The recombinant anaerobic, thermophilic host cell of claim 1, wherein a urease apo-enzyme in the host cell is activated by ureD, ureF, and ureG.

11. The recombinant anaerobic, thermophilic host cell of claim 1, wherein said host cell catalyzes the hydrolysis of urea to carbon dioxide and ammonia.

12. A method of producing ethanol comprising:
(a) culturing the recombinant anaerobic, thermophilic host cell of claim 1 in the presence of urea;
(b) contacting said anaerobic, thermophilic host cell with lignocellulosic biomass; and
(c) recovering the ethanol from the host cell culture.

13. The method of claim 12, wherein the host cell is cultured in the presence of at least about 0.5 g/L of urea.

14. The method of claim 13, wherein the host cell is cultured in the presence of at least about 1.0 g/L of urea.

15. The method of claim 12, wherein said host cell is of the genus Thermoanaerobacter or Thermoananerbacterium.

16. The method of claim 15, wherein said host is T. saccharolyticum.

17. The method of claim 12, wherein said host cell is co-cultured with a second anaerobic, thermophilic host strain.

18. The method of claim 17, wherein said second anaerobic, thermophilic host strain is C. thermocellum.

19. The method of claim 12, wherein said host is cultured in a medium having a pH range from about 4 to about 9.

20. The method of claim 19, wherein said host is cultured in a medium having a pH range from about 6 to about 8.

21. The method of claim 12, wherein said host cell produces increased ethanol titers with utilization of urea as a nitrogen source as compared to the levels of ethanol produced with utilization of complex additives or ammonium salts as a nitrogen source.

22. The method of claim 12, wherein said lignocellulosic biomass is selected from the group consisting of wood, corn, corn cobs, corn stover, corn fiber, sawdust, bark, leaves, agricultural and forestry residues, grasses such as switchgrass, cord grass, rye grass or reed canary grass, miscanthus, ruminant digestion products, municipal wastes, paper mill effluent, newspaper, cardboard, miscanthus, sugar-processing residues, sugarcane bagasse, agricultural wastes, rice straw, rice hulls, barley straw, cereal straw, wheat straw, canola straw, oat straw, oat hulls, stover, soybean stover, forestry wastes, recycled wood pulp fiber, paper sludge, sawdust, hardwood, softwood and combinations thereof.
